(19) United States
(12) Patent Application Publication
(10) Pub. No.: US 2025/0228577 A1
(43) Pub. Date: Jul. 17, 2025

(54) LITHOTRIPSY DEVICE FOR TUNNELING THROUGH VASCULAR OCCLUSIONS

(71) Applicant: Advanced Interventional Cardiovascular Solutions, LLC, Plano, TX (US)

(72) Inventors: Vijay Sitharam Ramanath, Plano, TX (US); David Scott Hazlitt, Lafayette, CO (US); Emily Rose Locker, Lafayette, CO (US); Joshua Eng-Morris, Denver, CO (US); Joshua K. Goetz, Denver, CO (US)

(21) Appl. No.: 19/017,736

(22) Filed: Jan. 12, 2025

Related U.S. Application Data
(60) Provisional application No. 63/620,667, filed on Jan. 12, 2024.

(51) Int. Cl. A61B 17/22; A61B 17/00

(52) U.S. Cl. CPC A61B 17/22022; A61B 17/00234; A61B 2017/00238; A61B 2017/00305; A61B 2017/00477; A61B 2017/00778; A61B 2017/00862; A61B 2017/00946; A61B 2017/22005; A61B 2017/22025; A61B 2017/22038; A61B 2017/22069

(57) ABSTRACT

A lithotripsy device for tunneling through vascular occlusions comprises a tunneling catheter including a tunneling catheter shaft having a distal end and a proximal end. A tunneling probe is disposed on the distal end of the tunneling catheter shaft. The tunneling probe has a pair of electrodes; each electrode being operatively connected to an electrical lead within the tunneling catheter shaft. The leads are configured for operative connection to an external pulse generator. Activation of an external pulse generator operatively connected to the leads causes repeated electrical discharges between the electrodes. When the electrodes are an in a fluid, the repeated electrical discharges produce repeated hydraulic shock waves in the fluid directed away from the electrical discharges. When the repeated hydraulic shock waves strike calcified tissue, the calcified tissue is broken into a modified plaque. The modified plaque has a lower resistance to mechanical dislocation that the original calcified tissue.

### CROSS-REFERENCE TO RELATED APPLICATIONS

This application claims benefit of U.S. Provisional Application No. 63/620,667, filed Jan. 12, 2024, entitled LITHOTRIPSY DEVICE FOR TUNNELING THROUGH VASCULAR OCCLUSIONS (Atty. Dkt. No. VJYR60-35815), which is incorporated by reference herein in its entirety.

### TECHNICAL FIELD

The following disclosure relates to a treatment system for percutaneous coronary angioplasty or peripheral angioplasty in which a tunneling device is used to make an initial pathway across an occluding lesion to allow subsequent crossing by a guidewire, dilation catheter, and/or other interventional equipment. Specifically, the tunneling device can be adapted to move through calcified occluding lesions.

### BACKGROUND

Treatment systems are known for percutaneous coronary angioplasty or peripheral angioplasty in which a dilation catheter is used to cross a lesion in order to dilate the lesion and restore normal blood flow in the vessel. In cases where the lesion is calcified or otherwise hardened, the dilation catheter can include a balloon capable of operating at high pressures to break the calcified or hardened plaque and push it back against the vessel wall. However, using a high-pressure balloon to break open calcified or hardened plaque carries the risk of causing trauma or injury to the artery or vessel wall, including perforation.

To reduce the chance of injury to the artery or vessel wall during angioplasty of calcified or hardened lesions, so-called intravascular lithotripsy (hereafter referred to as lithotripsy) treatment systems are employed wherein the balloon dilation catheter further includes a lithotripsy generator disposed within a fluid-filled balloon that forms a rapidly expanding and collapsing bubble within the balloon to form mechanical shock waves within the balloon which then travel through the balloon to adjacent tissue. The shock waves produced by such lithotripsy systems are conducted through the liquid, through the balloon, through the blood and vessel wall to the calcified lesion where the energy will break the hardened plaque without the application of excessive pressure by the balloon on the walls of the artery or vessel. Examples of such lithotripsy treatment systems are described in U.S. Pat. Nos. 9,072,534 and 11,020,135 assigned to Shockwave Medical, Inc.

The effective use of known lithotripsy treatment systems requires that the treatment device including the uninflated balloon and lithotripsy generator (collectively, the “lithotripsy dilation balloon”) be positioned within the occluded portion of the artery or vessel and at least partially filled with fluid (e.g., saline, contrast solution, etc.) before the shock wave generator can be activated to produce the desired therapeutic effect, i.e., the breaking up of calcified or hardened plaques using lithotripsy energy. However, in some cases the target plaques present in the artery or vessel have caused such extensive occlusion that the remaining passageway is too small in diameter or too convoluted to allow passage of the guidewire and/or lithotripsy dilatation catheter needed for the uninflated shock wave dilation balloon to cross the target plaque(s). Upon encountering such situations of extensive occlusion, it becomes necessary to first produce a pilot passageway through the target plaque(s) having both a sufficient diameter and a sufficiently straight pathway to allow passage of the guidewire needed for crossing by the uninflated lithotripsy dilation balloon. In some cases, the pilot passageway can be produced by forcibly pushing guidewires or catheters of suitable size against the occluding tissue using sufficient mechanical force to break through the occluding tissue until a pilot passageway of sufficient diameter and sufficient straightness is created. However, using mechanical force to push or poke wires through calcified or hardened plaques carries the risk of the occlusion deflecting the wire into the vessel wall or suddenly cracking and allowing the stressed guidewire to overshoot into the vessel wall, in either case possibly perforating all three layers of the artery or vessel wall or causing other trauma and injury to the vessel. In many instances, the wire is simply unable to cross these calcified occlusions and so the disease is unable to be treated percutaneously. A need therefore exists for a treatment system that can create a straight pilot passageway through a calcified or hardened lesion using low mechanical force that does not perforate the artery or vessel wall and minimizes trauma to the vessel.

### SUMMARY

In one aspect thereof, a treatment system is provided including a lithotripsy catheter having a lithotripsy tunneling probe on the distal end that can crack a calcified lesion while creating a new lumen (i.e., a “passageway lumen”) within the true lumen space (i.e., the “vessel lumen”) that can accommodate a guidewire therethrough. In one embodiment, the new lumen can accommodate a 0.014″ guidewire or larger passing therethrough.

In another aspect thereof, a treatment system is provided including a lithotripsy catheter having a lithotripsy tunneling probe on the distal end that can create a passageway through a calcified lesion while minimizing the amount of distal atheroemboli.

In yet another aspect thereof, a treatment system is provided including a lithotripsy catheter having a lithotripsy tunneling probe on the distal end that can create a passageway through a calcified lesion while remaining inside the vessel architecture and not perforating all three layers of the vessel wall.

In still another aspect thereof, a treatment system is provided including a support balloon or other support structure to aim the lithotripsy tunneling probe on the distal end of the lithotripsy catheter at the center of an occluding lesion and away from the vessel walls. In one embodiment thereof, a support balloon is disposed on the distal end of a support catheter. In another embodiment thereof, a distal end of the support catheter is the support structure. In yet another embodiment thereof, aiming the lithotripsy tunneling probe comprises positioning the distal end of the tunneling probe at a predetermined distance and orientation to the proximal face of the occluding lesion.

In a further aspect thereof, a treatment system is provided that includes a support catheter and a lithotripsy catheter having a lithotripsy tunneling probe on the distal end. The support catheter has a lumen sized to accommodate the lithotripsy catheter therein. The support catheter and lithotripsy catheter can be inserted into a vessel and the support catheter can be fixed relative to a body allowing the lithotripsy catheter to move relative to the body or be withdrawn from the body while the support catheter remains at a fixed position (i.e., forming a “base of operations”) in the vessel to then allow exchange of the lithotripsy catheter within the support catheter for a 0.035″ guidewire or smaller.

In a yet further aspect thereof, a treatment system is provided that includes a support catheter and a lithotripsy catheter having a lithotripsy tunneling probe on the distal end, wherein the support catheter that houses the lithotripsy catheter is compatible with a size 4 Fr (“French”) (or larger) sheath. In one embodiment, the system is configured for use from the tibial artery in a retrograde fashion. In another embodiment, the lithotripsy catheter is disposed inside the support catheter (mother/daughter) and the catheters are removably connected to one another by a screw interlocking system.

In a still further aspect thereof, a treatment system is provided that includes a support catheter and a lithotripsy catheter having a lithotripsy tunneling probe on the distal end, wherein the distal end of the support catheter is angulated to selectively aim the tunneling probe circumferentially or directionally around the proximal or distal (if performing a retrograde approach) end of the occlusion. In one embodiment, the angulated support catheter can be rotated within the vessel to change the aim of the tunneling probe. In another embodiment, the system further includes a fixture to allow selective rotation of the angulated support catheter within a vessel while maintaining the axial position of the support catheter within the vessel.

In another aspect thereof, a treatment system is provided that includes a support catheter and a lithotripsy catheter having a lithotripsy tunneling probe on the distal end, wherein the distal end of the support catheter is radio-opaque, the distal end of the lithotripsy catheter is radio-opaque, and the distal and of the support catheter is distinguishable from the distal end of the lithotripsy catheter under radiography.

In a further aspect, a lithotripsy device for tunneling through vascular occlusions comprises a tunneling catheter including a tunneling catheter shaft having a distal end and a proximal end. A tunneling probe is disposed on the distal end of the tunneling catheter shaft. The tunneling probe has a pair of electrodes; each electrode being operatively connected to an electrical lead within the tunneling catheter shaft. The leads are configured for operative connection to an external pulse generator. Activation of an external pulse generator operatively connected to the leads causes repeated electrical discharges between the electrodes. When the electrodes are in a fluid, the repeated electrical discharges produce repeated hydraulic shock waves in the fluid directed away from the electrical discharges. When the repeated hydraulic shock waves strike calcified tissue, the calcified tissue is broken into a modified plaque. The modified plaque has a lower resistance to mechanical dislocation than the original calcified tissue.

In one embodiment, the tunneling probe is configured with a raised rim on the distal end; and the electrodes are inset within the raised rim.

In another embodiment, the electrodes are configured in a concentric arrangement within the raised rim.

In yet another embodiment, the electrodes are configured in an opposing arcuate arrangement within the raised rim.

In still another embodiment, the tunneling probe is configured with a tapering conical face on the distal end; and the electrodes are configured in a spaced-apart arrangement on the tapering conical face.

In a further embodiment, the tapering conical face of the tunneling probe terminates in a flat face; and the electrodes are exposed on the flat face.

In a yet further embodiment, the tapering conical face of the tunneling probe terminates in a rounded end; and the electrodes are not exposed on the rounded end.

In a still further embodiment, the lithotripsy device further comprising a guidewire lumen formed through the catheter shaft and having an exit on the distal end. The tunneling probe is configured with a flat face on the distal end and the electrodes protrude above the flat face.

In another embodiment, the exit for the guidewire lumen is disposed in the center of the flat face of the distal end and the electrodes are configured in an opposing arcuate arrangement on opposite sides of the exit.

In yet another embodiment, the exit for the guidewire lumen is disposed offset from the center of the flat face of the distal end and the electrodes are configured as studs adjacent to one another on one side of the exit.

In still another embodiment, the exit for the guidewire lumen is disposed in the center of the flat face of the distal end and the electrodes are configured as studs on opposite sides of the exit.

In a further embodiment, the lithotripsy device further comprises a positioning balloon operatively attached to the tunneling catheter shaft near the distal end. The positioning balloon can be selectively inflated to position the catheter tunneling probe at a desired location within a cross section of the vessel.

In a yet further embodiment, the lithotripsy device further comprises a support catheter comprising a support catheter shaft having a distal end and a proximal end and defining an interior lumen configured to accommodate the tunneling catheter passing therethrough. When the support catheter is positioned with the distal end near the occlusion of calcified tissue, the proximal end can be affixed at an introduction site. When the support catheter is affixed at the introduction site, the tunneling catheter can be selectively inserted and withdrawn through the interior lumen to reach the occlusion of calcified tissue with the distal end having the tunneling probe.

In a still further embodiment, the support catheter further comprises a positioning balloon operatively attached to the support catheter shaft near the distal end. The positioning balloon can be selectively inflated to position the distal end of the support catheter at a desired location within a cross section of the vessel. When the tunneling catheter is introduced through the support catheter, the tunneling probe of the tunneling catheter will emerge from the support catheter at the desired location withing the cross section of the vessel.

In another embodiment, the lithotripsy device further comprises a pulse generator operatively connected to the electrical leads.

In a yet further aspect, a lithotripsy device for tunneling through vascular occlusions of calcified tissue in a liquid environment of a blood vessel is provided. The lithotripsy device comprises a tunneling catheter including a tunneling catheter shaft having a distal end and a proximal end. A tunneling probe is disposed on the distal end of the tunneling catheter shaft, the tunneling probe including a pair of electrodes, each electrode being operatively connected to an electrical lead within the tunneling catheter shaft, a raised rim on the distal end, wherein the electrodes are inset within the raised rim; and a flexible diaphragm affixed across the raised rim. The raised rim and the flexible diaphragm define a probe cavity containing the electrodes, the probe cavity being fluidly isolated from a fluid environment external to the probe cavity. The leads are configured for operative connection to an external pulse generator. Activation of an external pulse generator operatively connected to the leads causes repeated electrical discharges between the electrodes. When the probe cavity contains a working fluid, the repeated electrical discharges produce repeated primary shock waves in the working fluid that repeatedly vibrate the flexible diaphragm. The repeated vibration of the flexible diaphragm produces repeated secondary shock waves propagated into the fluid environment external to the probe cavity. When the repeated secondary shock waves strike calcified tissue in the fluid environment, the calcified tissue breaks into a modified plaque. The modified plaque has a lower resistance to mechanical dislocation that the original calcified tissue.

In one embodiment, the flexible diaphragm is formed of a metal or metal alloy.

In another embodiment, the flexible diaphragm is formed of a plastic, polymer, elastomer or other non-metallic material.

In yet another embodiment, the lithotripsy device further comprises a fluid lumen formed within the tunneling catheter shaft and fluidly connecting the probe cavity to a source of fresh working fluid for circulating the fresh working fluid through the probe cavity during the repeated electrical discharges.

In a still further aspect, a method for using a lithotripsy device for tunneling through vascular occlusions of calcified tissue in a liquid environment of a blood vessel comprises the following steps: providing a lithotripsy tunneling catheter and a support catheter; introducing a guidewire into a fluid-filled vascular system and guiding the guidewire into a blood vessel having a calcified tissue occlusion; introducing the support catheter into the vascular system over the guidewire to the location of the calcified tissue occlusion; inflating a positioning balloon on the support catheter in the blood vessel; affixing the support catheter at introduction site to minimize axial movement of support catheter in the vessel; withdrawing the guidewire from the lumen of the support catheter; introducing the tunneling catheter through support catheter to location of calcified tissue occlusion and positioning the tunneling probe in the blood vessel adjacent the calcified tissue; producing electrical discharges between electrodes on the tunneling probe to create hydraulic shock waves in the fluid of the vessel; directing the shock waves produced by the electrical discharges through the fluid in the vessel into the calcified tissue of occlusion to break up the calcified tissue into a modified plaque; and channeling through the modified plaque with reduced mechanical force to create a channel through the calcified tissue of the occlusion along a desired line.

In another aspect, a lithotripsy catheter is provided with a sealed-chamber “drum” type tunneling probe. The catheter end effector is comprised of a rigid outer “can” (metal hypo tube and metal washer that are laser welded together), two electrical leads with exposed ends (electrodes), multi-lumen shaft (fluid inlet, fluid outlet, and two electrical lead lumens), and membrane (polymer with low acoustic impedance that is thermally or adhesively attached to the “can”). The chamber is filled with fluid (e.g., saline or saline-contrast mixture) that circulates through the multi-lumen shaft to evacuate air bubbles and cool the end effector components. Energy delivered by the generator to the exposed ends of the electrical leads (i.e., electrodes) creates electrical discharges and subsequent fluid cavitation within the chamber. Hydraulic shock waves form and are focused axially forward by an external rigid (metal) tube with high acoustic impedance through a membrane with low acoustic impedance and subsequently propagate into calcified tissue. Repeated exposure of the calcified tissue to hydraulic shock waves creates modified plaque that is more susceptible to dislocation than the original calcified tissue.

In another embodiment, the lithotripsy catheter is provided with a sealed-chamber “drum” type tunneling probe. The catheter end effector is comprised of a rigid outer “can” (metal inner and outer hypo tubes and two metal washers that are laser welded together), two electrical leads with exposed ends (electrodes), multi-lumen shaft (fluid inlet, fluid outlet, and two electrical lead lumens), and membrane (polymer with low acoustic impedance that is thermally, mechanically, or adhesively attached to the “can”). The chamber is filled with fluid (e.g., saline or saline-contrast mixture) that circulates through the multi-lumen shaft to evacuate air bubbles and cool the end effector components. Energy delivered by the generator to the exposed ends of the electrical leads (i.e., electrodes) creates electrical discharges and subsequent fluid cavitation within the chamber. Hydraulic shock waves form and are focused axially forward by an external rigid (metal) tube with high acoustic impedance through a membrane with low acoustic impedance and subsequently propagate into calcified tissue. Repeated exposure of the calcified tissue to hydraulic shock waves creates modified plaque that is more susceptible to dislocation than the original calcified tissue.

In yet another embodiment, a lithotripsy catheter is provided with a sealed-chamber “drum” type tunneling probe. The catheter end effector is comprised of a rigid outer “can” (metal inner and outer hypo tubes and metal washer that are laser welded together), two electrical leads with exposed ends (electrodes), multi-lumen shaft (fluid inlet, fluid outlet, and two electrical lead lumens), and membrane (polymer with low acoustic impedance that is thermally, mechanically, or adhesively attached to the “can”). The chamber is filled with fluid (e.g., saline or saline-contrast mixture) that circulates through the multi-lumen shaft to evacuate air bubbles and cool the end effector components. Energy delivered by the generator to the exposed ends of the electrical leads (i.e., electrodes) creates electrical discharges and subsequent fluid cavitation within the chamber. Hydraulic shock waves form and are focused axially forward by an external rigid (metal) tube with high acoustic impedance through a membrane with low acoustic impedance and subsequently propagate into calcified tissue. Repeated exposure of the calcified tissue to hydraulic shock waves creates modified plaque that is more susceptible to dislocation than the original calcified tissue.

In yet another aspect, a lithotripsy catheter is provided with a sealed-chamber “drum” type tunneling probe. The catheter end effector is comprised of a rigid outer tube (metal hypo tube that is adhesively, mechanically, or thermally attached to the membrane and shaft), two electrical leads with exposed ends (electrodes), multi-lumen shaft (fluid inlet, fluid outlet, and two electrical lead lumens), and “sock-type” membrane (polymer with low acoustic impedance that is thermally adhesively attached to the shaft). The chamber is filled with fluid (e.g., saline or saline-contrast mixture) that circulates through the multi-lumen shaft to evacuate air bubbles and cool the end effector components. Energy delivered by the generator to the exposed ends of the electrical leads (i.e., electrodes) creates electrical discharges and subsequent fluid cavitation within the chamber. Hydraulic shock waves form and are focused axially forward by an external rigid (metal) tube with high acoustic impedance through a membrane with low acoustic impedance and subsequently propagate into calcified tissue. Repeated exposure of the calcified tissue to hydraulic shock waves creates modified plaque that is more susceptible to dislocation than the original calcified tissue.

In a still further aspect, a lithotripsy catheter is provided with a sealed-chamber “drum” type tunneling probe. The catheter end effector is comprised of a rigid outer “can” (metal inner and outer hypo tubes), single-lumen shaft, and membrane (polymer with low acoustic impedance that is thermally, mechanically, or adhesively attached to the “can”). The shaft and chamber are filled with fluid (e.g., saline or saline-contrast mixture) that conducts high frequency hydraulic pulsatile pressure waves. An external generator uses compressed CO₂ to create pulsatile waves through a diaphragm in the handle of the device. Hydraulic shock waves form and are focused axially forward by an external rigid (metal) tube with high acoustic impedance through a membrane with low acoustic impedance and subsequently propagate into calcified tissue. Repeated exposure of the calcified tissue to hydraulic shock waves creates modified plaque that is more susceptible to dislocation than the original calcified tissue.

### BRIEF DESCRIPTION OF THE DRAWINGS

For a more complete understanding, reference is now made to the following description taken in conjunction with the accompanying Drawings in which:

FIG. 1 shows a lithotripsy catheter with a cannon/bumper type tunneling probe having concentric electrodes without a central lumen in accordance with one aspect. The probe can include a rigid outer tube with internal electrodes/emitter that are positioned proximally relative to distal end of probe. The rigid outer tube directs shockwaves distally (end emitting) and the inset emitter helps the user position catheter at an optimal distance from the calcified lesion for therapeutic effect;

FIG. 2 shows another lithotripsy catheter with a cannon/bumper tunneling probe having arched electrodes with a central lumen in accordance with another embodiment;

FIG. 3 shows a lithotripsy catheter with a taper end tunneling probe with a flat tip and without a central lumen having narrow lateral electrodes with exposed ends on the flat tip in accordance with another aspect;

FIG. 4 shows another lithotripsy catheter with a taper end tunneling probe with a round tip without a central lumen having wide lateral electrodes in accordance with another embodiment;

FIG. 5 shows yet another lithotripsy catheter with a taper end tunneling probe with a flat tip and without a central lumen having wide lateral electrodes with exposed ends on the flat tip in accordance with another embodiment;

FIG. 6 shows a lithotripsy catheter with an over-the-wire tunneling probe having a central lumen with arched electrodes protruding from the end in accordance with another aspect;

FIG. 7 shows another lithotripsy catheter with an over-the-wire tunneling probe having a D-shaped lumen with adjacent stud electrodes protruding from the end in accordance with another embodiment;

FIG. 8 shows yet another lithotripsy catheter with an over-the-wire tunneling probe having a central lumen with opposed electrodes protruding from the end in accordance with another embodiment;

FIG. 9 shows a lithotripsy catheter with a sealed-chamber “drum” type tunneling probe in accordance with another aspect;

FIG. 10 is a cross-sectional view of the sealed-chamber “drum” type tunneling probe of FIG. 9;

FIG. 11 shows yet another lithotripsy catheter having a positioning balloon in accordance with another aspect;

FIGS. 12A, 12B and 12C illustrate the use of the lithotripsy catheter having a positioning balloon of FIG. 11 in a blood vessel totally occluded by calcified tissue;

FIGS. 12D and 12E illustrate the use of the lithotripsy catheter having a positioning balloon of FIG. 11 in a blood vessel having a lumen that is substantially narrowed by calcified tissue;

FIG. 13 shows a positioning support catheter in accordance with another aspect for use in connection with a lithotripsy tunneling catheter;

FIGS. 14A, 14B, 14C and 14D illustrate the use of the positioning support catheter of FIG. 13 with a lithotripsy tunneling probe and a guidewire in a blood vessel totally occluded by calcified tissue;

FIGS. 14E and 14F illustrate the use of the positioning support catheter of FIG. 13 with a lithotripsy tunneling probe and a guidewire in a blood vessel having a lumen that is substantially narrowed by calcified tissue;

FIG. 15 shows an alternative centering support catheter in accordance with another aspect for use in connection with a lithotripsy tunneling catheter;

FIGS. 16A and 16B illustrate the use of the alternative positioning support catheter of FIG. 15 with a lithotripsy tunneling probe in a blood vessel occluded by calcified tissue;

FIG. 17 shows a straight support catheter in accordance with yet another aspect for use in connection with a lithotripsy tunneling catheter;

FIG. 18 shows a curved support catheter in accordance with still another aspect for use in connection with a lithotripsy tunneling catheter;

FIG. 19 shows a block diagram of a method for the use of a lithotripsy device for tunneling a crossing through vascular occlusions formed of calcified tissue;

FIG. 20 is a front perspective view of a lithotripsy catheter with a sealed-chamber “drum” type tunneling probe in accordance with another embodiment;

FIG. 21 is a front perspective view of the lithotripsy catheter of FIG. 20 with the front membrane retainer shown transparently for purposes of illustration;

FIG. 22 is a partial cross-sectional side view of the lithotripsy catheter of FIG. 20;

FIG. 23 is a front perspective view of a lithotripsy catheter with a sealed-chamber “drum” type tunneling probe in accordance with another embodiment;

FIG. 24 is a front perspective view of the lithotripsy catheter of FIG. 23 with the front membrane retainer shown transparently for purposes of illustration;

FIG. 25 is a partial cross-sectional side view of the lithotripsy catheter of FIG. 23;

FIG. 26 is a partial cross-sectional side view of a lithotripsy catheter with a sealed-chamber “drum” type tunneling probe in accordance with another embodiment;

FIG. 27 shows a prototype tunneling catheter in accordance with the embodiment of FIG. 26;

FIG. 28 shows a prototype tunneling catheter in accordance with the embodiment of FIGS. 29-33;

FIG. 29 is a front view of a lithotripsy catheter with a sealed-chamber “sock” type tunneling probe in accordance with another embodiment;

FIG. 30 is a front perspective view of the lithotripsy catheter of FIG. 29;

FIG. 31 is a cross-sectional side view of a “sock” type membrane suitable for use in the lithotripsy catheter of FIG. 29;

FIG. 32 is an enlarged partial cross-sectional side view of the lithotripsy catheter of FIG. 29;

FIG. 33 is another partial cross-sectional side view of the lithotripsy catheter of FIG. 29;

FIG. 34 is a cross-sectional side view of a lithotripsy catheter with a sealed-chamber “sock” type tunneling probe suitable for use with an external high-frequency hydraulic pulsatile pressure wave generator; and

FIG. 35 is a front perspective view of the lithotripsy catheter of FIG. 34, wherein the length of the catheter has been shortened for purposes of illustration.

### DETAILED DESCRIPTION

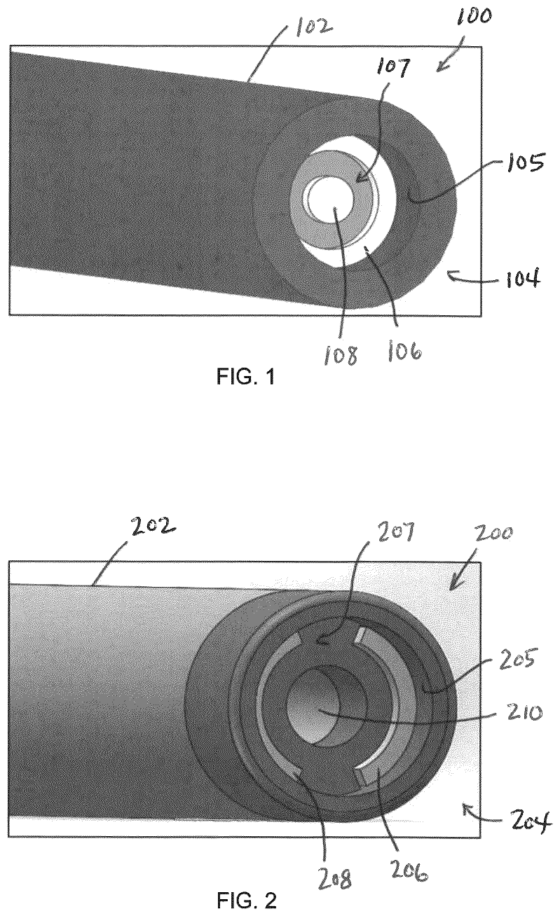

Referring to FIGS. 1-11, various aspects of the lithotripsy device for tunneling vascular occlusions are provided. As a baseline for the electrical design aspects of the system, electrical pulse generator systems derived from conventional electrohydraulic lithotripsy (“EHL”) equipment are suitable. However, new configurations for the probes and new designs for the electrical pulse waveforms and resulting hydraulic pressure shock waves are provided to optimize the system for tunneling through calcified and hardened lesions found occluding the vascular system while minimizing the chance of injury to the actual arteries or vessels.

Some embodiments of the lithotripsy device for tunneling through vascular occlusions include a tunneling catheter (including tunneling shaft and tunneling probe) having an outer diameter (“OD”) of 6 French (“Fr”) or less and configured to be compatible with (i.e., pass through) a 6 Fr inner diameter (“ID”) introducer sheath. Some embodiments of the lithotripsy device include a tunneling catheter with shaft and probe having an OD of 4 Fr or less and configured to be compatible with a 4 Fr ID introducer sheath. Some embodiments of the lithotripsy device include a support catheter and a tunneling catheter, wherein the support catheter has an ID of 6 Fr or less and is configured to be compatible with an introducer sheath having an ID of 8 Fr or less, and wherein the tunneling catheter (including tunneling shaft and tunneling probe) is configured to be compatible with the 6 Fr or less ID of the support catheter. Further embodiments of the lithotripsy device include a support catheter and a tunneling catheter, wherein the support catheter is configured to be compatible with an introducer sheath having an ID of 6 Fr or less, and wherein the tunneling catheter is configured to be compatible with the ID of the support catheter.

To be clear, the tunneling lithotripsy devices of the current invention are not intended for significantly dilating the calcified or hardened lesions to restore blood flow. Even in cases where the tunneling lithotripsy device includes a balloon of some sort, the balloon is not a dilation balloon. Rather, the tunneling lithotripsy devices of the current invention operate to crack, break, pulverize or otherwise soften the calcified or hardened lesions only to facilitate the crossing of a guidewire needed for subsequent effective dilation of the lesion by a separate dilation balloon. The separate dilation balloon can be, but is not limited to, a conventional high pressure dilation balloon or a shock wave generator type dilation balloon.

Referring now specifically to FIG. 1, there is illustrated a lithotripsy tunneling catheter in accordance with a first aspect. The tunneling catheter 100 includes a catheter shaft 102 and tunneling probe 104 on the distal end. The tunneling probe 104 includes a raised or protruding rim 105 defining an electrode cavity 107 therewithin. Concentric electrodes 106 and 108 are positioned on the base of the electrode cavity 107 facing the open distal end. In this manner, the concentric electrodes 106, 108 are recessed a predetermined distance from the distal end of the probe 104. In some embodiments, the protruding rim 105 of the probe 104 is formed by a rigid outer tube and the internal electrodes/emitter 106, 108 are positioned proximally relative to the distal end of probe. The rigid outer tube directs shockwaves distally (end emitting) and the inset emitter helps the user position catheter at an optimal distance from the calcified lesion for therapeutic effect. In the illustrated embodiment, the tunneling catheter 100 does not include a communicating central lumen. Unless otherwise indicated, the term “communicating” lumen means a lumen that is open on the distal end of the catheter shaft to allow the passage of a guidewire therethrough and/or to allow the passage of irrigation or suction. The tunneling catheter 100 further includes electrical leads (not illustrated) operably connected to the electrodes 106, 108 and configured for operably connection to an external pulse generator.

By activating a pulse generator connected to the electrodes 106, 108, a series of brief electrical discharges are initiated between the electrodes. Each electrical discharge vaporizes the surrounding fluids (e.g., blood plasma) to form a rapidly expanding and collapsing gas bubble at the site of the discharge. As the gas bubbles expand and collapse, hydraulic shock waves are formed that propagate from the discharge site through the fluid to adjacent tissue. These shock waves are generally harmless to soft (i.e., flexible) tissue, but can crack apart calcified or other hardened tissues such as plaque lesions. In preferred embodiments, the pulse generator is operably connected to a pulse controller that can selectively modify the power, frequency, duration, waveform and other characteristics of the electrical discharges initiated by the pulse generator to produce shock waves appropriate for tunneling through different types of lesions. In some embodiments using a pulse controller, the pulse generator and pulse controller are separate units, whereas in other embodiments, they are combined into a single unit. After the calcified or hardened occluding tissue is broken down by the shock waves, the tunneling catheter 100 can be advanced into the softened tissue to form a channel through the occlusion. Alternatively, after softening the occluding tissue with shock waves, the tunneling catheter 100 can be withdrawn and replaced by a guidewire to form a channel into the softened material of the occlusion. After the desired channel is formed through the calcified material, the tunneling catheter 100 can be withdrawn and dilation of the occlusion can be performed by a separate dilation balloon.

Referring now specifically to FIG. 2, there is illustrated another lithotripsy tunneling catheter in accordance with a second aspect. The tunneling catheter 200 includes a catheter shaft 202 and tunneling probe 204 on the distal end. The tunneling probe 204 includes a raised or protruding rim 205 forming an electrode cavity 207 therewithin. Arched electrodes 206 and 208 are positioned on the base of the electrode cavity 207 on opposite sides of a communicating central lumen 210. The central lumen 210 can be operably connected to the proximal end of the shaft 202 to allow passage of a guidewire therethrough. In some embodiments, the central lumen 210 can allow fluid irrigation and/or fluid suction to be provided in the region adjacent the tunneling probe 204. The tunneling catheter 200 further includes electrical leads (not illustrated) operably connected to the electrodes 206, 208 and configured for connection to an external pulse generator. By activating the pulse generator, electrical discharges are produced between the electrodes 206 and 208 to form hydraulic shock waves that propagate from the discharge site and tunnel into the occluding calcified tissue by breaking it up as previously described. After the desired channel is formed through, or partially through, the calcified material, the tunneling catheter 200 can be withdrawn and dilation of the occlusion can be performed by a separate dilation balloon.

The tunneling catheters 100 and 200 both have a “cannon/bumper” type tunneling probe configuration wherein a rigid outer tube with electrodes are recessed within the electrode cavity created by the raised rim on the end of the probe. The raised rim propagates the shock waves from the electrode cavity in a generally axial direction (i.e., parallel to the central axis of the catheter). Further, the raised rim prevents the electrodes from directly contacting the occluding tissue. Further still, the raised rim ensures that the shock wave is produced at a predetermined distance from the occlusion (presuming the distal end of the probe is touching the occlusion or disposed at a predetermined distance from the occlusion). Advantages of the cannon/bumper style tunneling probe include, but are not limited to, good directionality of the shock wave, a consistent distance between the electrodes and the lesion if the distal end of the catheter is pressed against the lesion, and the ability to use the central lumen 210 for a guidewire or irrigation/suction (catheter 200 only). Disadvantages of the cannon/bumper style probe include that electrical arcing in blood is untested and may present functional or safety (thrombus) risks to patient under some conditions, and that interaction between guidewire (if present) and electrodes may produce safety risks. Note that such disadvantages, if present, may be mitigated or overcome by developing appropriate protocols for use and/or additional features.

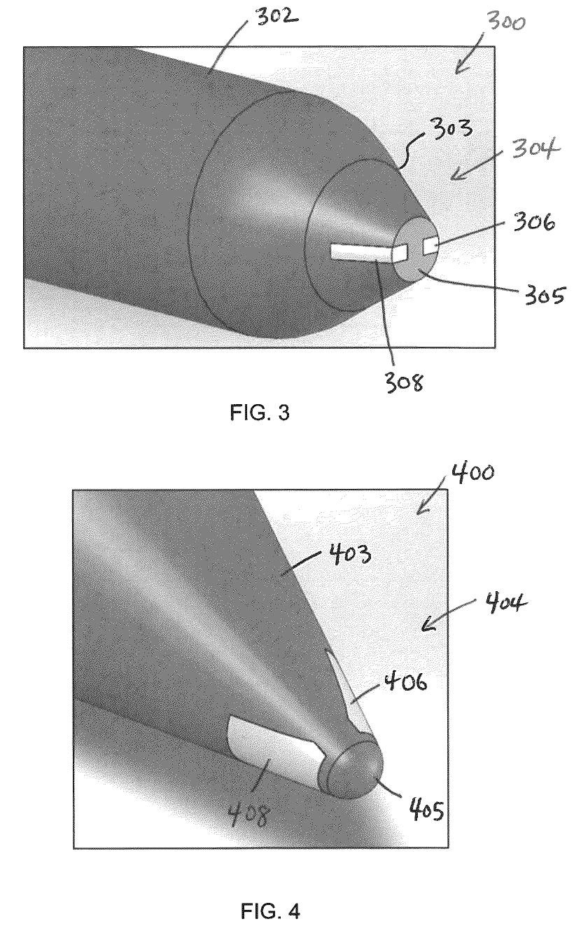

Referring now specifically to FIG. 3, there is illustrated a lithotripsy tunneling catheter in accordance with a third aspect. The tunneling catheter 300 includes a catheter shaft 302 and exposed tunneling probe 304 having a tapered distal end 303 a flat tip 305. The tunneling probe 304 includes narrow lateral electrodes 306, 308 with exposed ends on the flat tip 305. There is no communicating central lumen. The tunneling catheter 300 further includes electrical leads (not illustrated) operably connected to the electrodes 306, 308 and configured for connection to an external pulse generator. By activating the pulse generator, electrical discharges are produced between the electrodes 306 and 308. When the flat tip 305 is spaced apart from a lesion, the electrical discharges from the electrodes 306, 308 can travel through the surrounding fluid to produce shock waves as previously described that propagate away from the probe. When the flat tip 305 is pressed against a lesion, at least a portion of the electrical discharge can travel between the exposed ends of the electrodes 306, 308 through the contacted portion of the lesion, thus directly destroying the contacted portion via cauterization. Using lithotripsy and heat energy, the probe 304 tunnels into the occluding calcified tissue by breaking it up as previously described. After the desired channel is formed through, or partially through, the calcified material, the tunneling catheter 300 can be withdrawn and dilation of the occlusion can be performed by a separate dilation balloon.

Referring now specifically to FIG. 4, there is illustrated a lithotripsy tunneling catheter in accordance with a fourth aspect. The tunneling catheter 400 includes a catheter shaft (not shown) and exposed tunneling probe 404 having a tapered distal end 403 and a round tip 405. The tunneling probe 404 includes wide lateral electrodes 406, 408 disposed on the tapered end 403 that do not extend onto the round tip 405. There is no communicating central lumen. The tunneling catheter 400 further includes electrical leads (not illustrated) operably connected to the electrodes 406, 408 and configured for connection to an external pulse generator. By activating the pulse generator, electrical discharges are produced between the electrodes 406 and 408 to produce shock waves as previously described that propagate away from the probe into the occluding tissue. Using lithotripsy, the probe 404 tunnels into the occluding calcified tissue by breaking it up as previously described. After the desired channel is formed through, or partially through, the calcified material, the tunneling catheter 400 can be withdrawn and dilation of the occlusion can be performed by a separate dilation balloon.

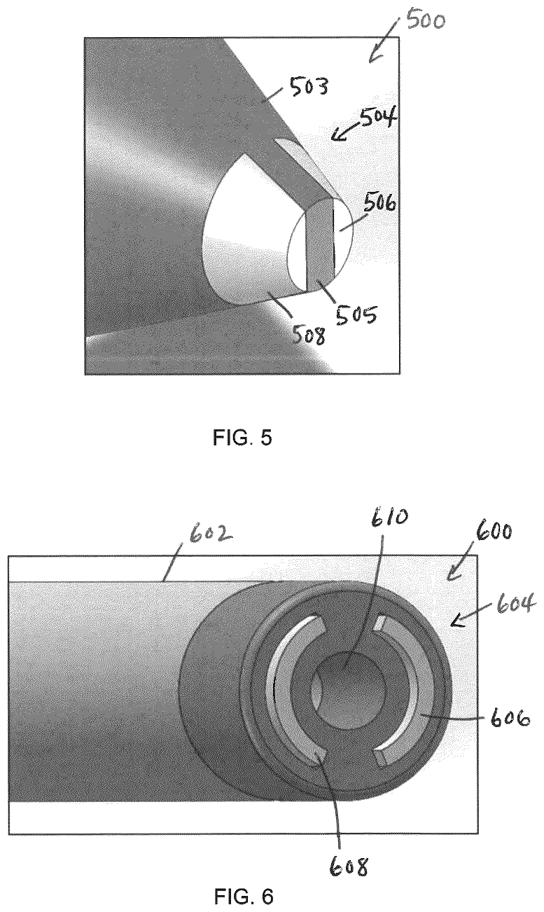

Referring now specifically to FIG. 5, there is illustrated a lithotripsy tunneling catheter in accordance with a fifth aspect. The tunneling catheter 500 includes a catheter shaft (not shown) and exposed tunneling probe 504 having a tapered distal end 503 and a flat tip 505. The tunneling probe 504 includes wide lateral electrodes 506, 508 with exposed ends on the flat tip 505. There is no communicating central lumen. The tunneling catheter 500 further includes electrical leads (not illustrated) operably connected to the electrodes 506, 508 and configured for connection to an external pulse generator. By activating the pulse generator, electrical discharges are produced between the electrodes 506 and 508. Similar to probe 300, when the flat tip 505 is spaced apart from a lesion, the electrical discharges from the electrodes 506, 508 can travel through the surrounding fluid to produce shock waves propagating away from the probe, but when the flat tip is pressed against a lesion, at least a portion of the electrical discharge can travel between the exposed ends of the electrodes through the contacted portion of the lesion. Using shock waves and heat energy, the probe 504 tunnels into the occluding calcified tissue by breaking it up as previously described. After the desired channel is formed through, or partially through, the calcified material, the tunneling catheter 500 can be withdrawn and dilation of the occlusion can be performed by a separate dilation balloon

The tunneling catheters 300, 400 and 500 all have a “taper” type tunneling probe configuration wherein the distal end of the tunneling probe is tapered to improve the ability to mechanically push through a lesion after cracking by the lithotripsy shock waves. Advantages of the taper style tunneling probe include, but are not limited to, a simple design without multiple lumens and the ability to mechanically open a channel after cracking of calcifications. Disadvantages of the taper style probe include reduced directionality (i.e., of the shock waves), that the small overall scale of the probe diminishes the value of the tapered tip, the tapered tip may increase perforation risk, and (as previously mentioned) that electrical arcing in blood is untested and may present functional or safety risks to patient under some conditions. Note that such disadvantages, if present, may be mitigated or overcome by developing appropriate protocols for use and/or additional features.

Referring now specifically to FIG. 6, there is illustrated another lithotripsy tunneling catheter in accordance with a sixth aspect. The tunneling catheter 600 includes a catheter shaft 602 and protruding tunneling probe 604 on the distal end. The tunneling probe 604 includes arched electrodes 606, 608 positioned on opposite sides of a central lumen 610. The central lumen 610 can run over a guidewire (not shown). In one embodiment, the guidewire can be a 0.014″ (inch) guidewire. The tunneling catheter 600 further includes electrical leads (not illustrated) operably connected to the electrodes 606, 608 and configured for connection to an external pulse generator. By activating the pulse generator, electrical discharges are produced between the electrodes 606 and 608 to create shock waves that propagate from the probe 604 through the surrounding fluid to crack or otherwise break down calcified or inflexible occlusions. After the desired channel is formed through, or partially through, the calcified material, the tunneling catheter 600 can be withdrawn and dilation of the occlusion can be performed by a separate dilation balloon.

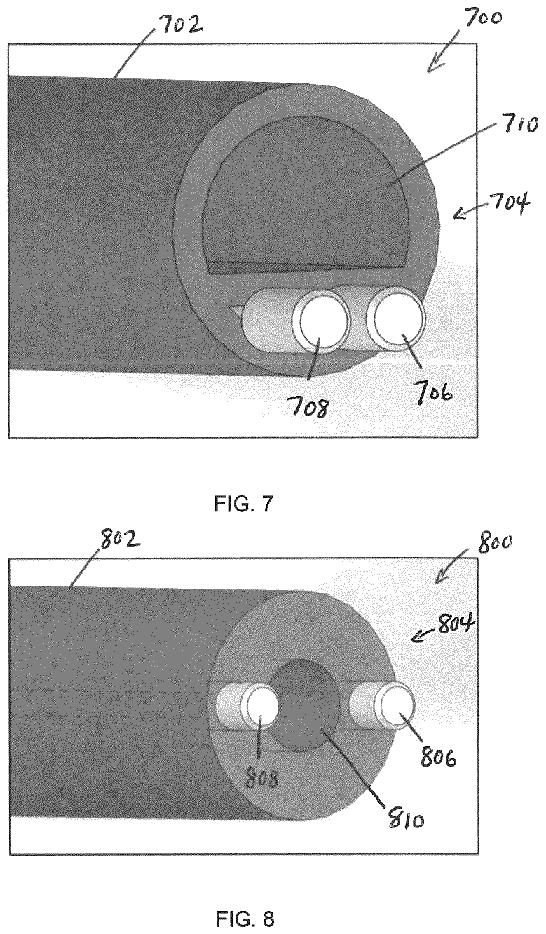

Referring now specifically to FIG. 7, there is illustrated another lithotripsy tunneling catheter in accordance with a seventh aspect. The tunneling catheter 700 includes a catheter shaft 702 and protruding tunneling probe 704 on the distal end. The tunneling probe 704 includes a pair of side-by-side, stud-type electrodes 706 and 708, which are offset from a lumen 710 disposed to one side of the catheter shaft 702. In the illustrated embodiment, the offset lumen 710 is “D” shaped (in cross section), however, in other embodiments the offset lumen can have a different shape. The offset lumen 710 can run over a guidewire (not shown). In one embodiment, the guidewire can be a 0.014″ (inch) guidewire. The tunneling catheter 700 further includes electrical leads (not illustrated) operably connected to the electrodes 706, 708 and configured for connection to an external pulse generator. By activating the pulse generator, electrical discharges are produced between the electrodes 706 and 708 to create shock waves that propagate from the probe 704 through the surrounding fluid to crack or otherwise break down the occluding hardened tissue. After the desired channel is formed through, or partially through, the calcified material, the tunneling catheter 700 can be withdrawn and dilation of the occlusion can be performed by a separate dilation balloon.

Referring now specifically to FIG. 8, there is illustrated another lithotripsy tunneling catheter in accordance with an eighth aspect. The tunneling catheter 800 includes a catheter shaft 802 and protruding tunneling probe 804 on the distal end. The tunneling probe 804 includes a pair of stud-type electrodes 806, 808 disposed on opposite sides of a central lumen 810. The central lumen 810 can run over a guidewire (not shown). In one embodiment, the guidewire can be a 0.014″ (inch) guidewire but also can accommodate up to a 0.035″ (inch) guidewire. The tunneling catheter 800 further includes electrical leads (not illustrated) operably connected to the electrodes 806, 808 and configured for connection to an external pulse generator. By activating the pulse generator, electrical discharges are produced between the electrodes 806 and 808 to create shock waves that propagate from the probe 804 through the surrounding fluid to crack or otherwise break down the occluding hardened tissue. After the desired channel is formed through, or partially through, the calcified material, the tunneling catheter 600 can be withdrawn and dilation of the occlusion can be performed by a separate dilation balloon.

The tunneling catheters 600, 700 and 800 all have an “over-the-wire” type tunneling probe configuration wherein the respective electrodes extend beyond the distal end of the shaft and the probe runs over a guidewire. In some embodiments, the central lumen can allow fluid irrigation or fluid suction to be provided in the region adjacent the tunneling probe. In comparison to the cannon/bumper type probes 100 and 200 having a raised rim at the end of the probe, the over-the-wire type probes 600, 700 and 800 do not have a raised rim, and thus do not have an electrode cavity to focus the shock waves axially. Therefore, the over-the-wire type tunneling probes 600, 700 and 800 can propagate shock waves radially away from the probe as well as axially. Other advantages of the over-the-wire style tunneling probe include, but are not limited to, faster and easier exchange of the lithotripsy catheter for another device, e.g., a high-pressure dilation catheter, a shock wave dilation catheter, etc. Further advantages include so-called “inchworm” functionality, wherein the tunneling probe is used to soften a first portion of the lesion, followed by using a guidewire to channel the first softened portion, followed by incrementally advancing the tunneling probe into the newly created channel to soften an incrementally further second portion of the lesion, and repeating this process until the desired channel is completed through the lesion. As described above, the central lumen can also facilitate fluid irrigation or fluid suction in the affected region adjacent the tunneling probe. Disadvantages of the over-the-wire style tunneling probe include are similar to those of the cannon/bumper style probe, include that electrical arcing in blood is untested and may present functional or safety risks to patient under some conditions, and that interaction between guidewire and electrodes may produce safety risks. An additional disadvantage is the small diameter of guidewire that can be accommodated in the guidewire lumen given the overall dimensional limitation of the tunneling catheter. Note that such disadvantages, if present, may be mitigated or overcome by developing appropriate protocols for use and/or additional features.

In some embodiments, the respective lumens 610, 710 and 810 of the respective over-the-wire catheters 600, 700 and 800 can further include two separate fluid passages in the lumen. When two separate fluid passages are provided, one fluid passage can be used for fluid inlet and the other fluid passage can be used for fluid withdrawal. In some embodiments, the fluid inlet passage can be used for irrigation (e.g., using saline) of the area directly surrounding the tunneling probe before, during or after lithotripsy operations. In some embodiments, the fluid withdrawal passage can be used for suction (e.g., of blood or saline containing lithotripsy debris) of the area directly surrounding the tunneling probe before, during or after lithotripsy operations. In some embodiments, simultaneous irrigation and suction can be provided to provide controlled circulation of fluid through the lithotripsy area to remove heated fluids and/or debris caused by the electrical discharges during the lithotripsy operation.

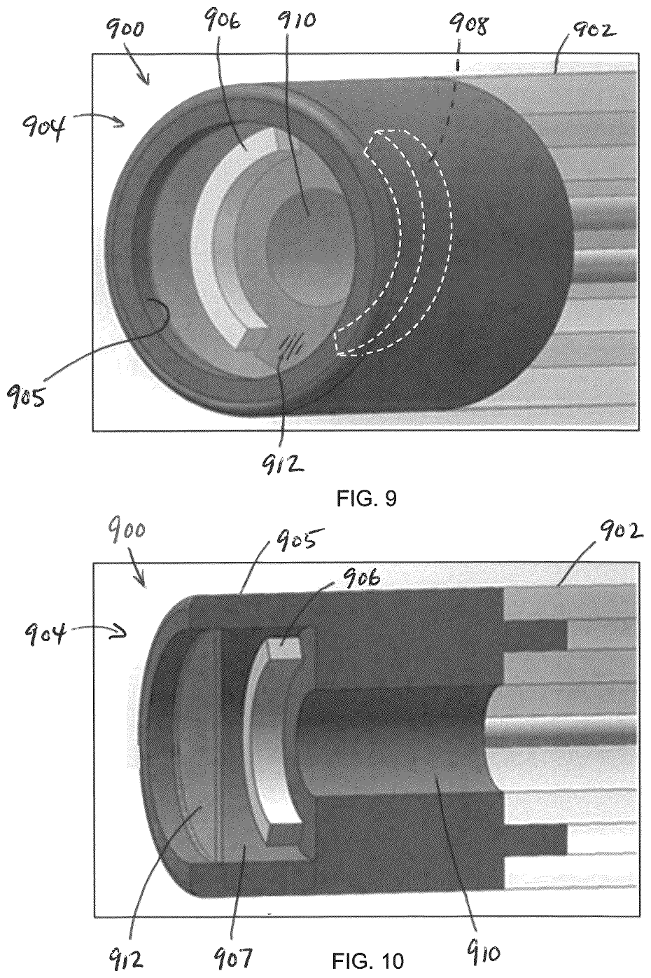

Referring now specifically to FIGS. 9 and 10, there is illustrated another lithotripsy tunneling catheter in accordance with a ninth aspect. The tunneling catheter 900 includes a catheter shaft 902 and tunneling probe 904 on the distal end. Similar to the probe 200 previously described, the tunneling probe 904 further includes a raised or protruding rim 905 forming an electrode cavity 907 therewithin, and electrodes 906 and 908 (shown in phantom line) are positioned on the base of the electrode cavity. However, unlike the probe 200, the tunneling probe 904 further includes a flexible diaphragm 912 affixed across the distal end of the probe to fluidly isolate the electrode cavity 907 from the vessel, forming a “drum” configuration. In other words, the electrode cavity 907 can be filled with one fluid, e.g., saline or contrast solution, that is isolated from the fluid in the vessel, e.g., blood plasma. The tunneling catheter 900 further includes electrical leads (not illustrated) operably connected to the electrodes 906, 908 and configured for connection to an external pulse generator.

By activating the pulse generator connected to the probe 904, electrical discharges are produced between the electrodes 906 and 908 to create primary shock waves propagated into the fluid within the electrode cavity 907. These primary shock waves impinge on one side of the diaphragm 912, causing it to flex or vibrate against the fluid of the vessel on the other side, and thus producing secondary shock waves propagated into the fluid of the vessel while still maintaining fluid isolation between the two fluids. Since the fluid in the electrode cavity 907 is always isolated from the vessel, any possible undesirable thermal or physical effects of electrical discharge directly in a blood-filled vessel are avoided. The diaphragm 912 can be made from any fluid-impermeable flexible material, including elastomers, polymers and thin metals. Depending on the stiffness of the material used for the diaphragm 912, the secondary shock waves can be enhanced or attenuated relative to the primary shock waves; however, potential attenuation can be compensated for by increasing the power of the primary shock waves or by otherwise modifying the electronic discharge pulses using the pulse controller. Depending on the respective acoustic impedances of the respective fluids in the electrode cavity 907 and vessel, the secondary shock waves can be attenuated relative to the primary shock waves; however, such attenuation can be compensated for by increasing the power of the primary shock waves or by otherwise modifying the electronic discharge pulses using the pulse controller.

In some embodiments, the tunneling catheter 904 can further includes at least one lumen 910 communicating with the sealed electrode cavity 907. The lumen 910 can be used to fill the electrode cavity 907 with fluid. In some embodiments, two lumens, e.g., 910′ and 910″ can be provided communicating with the sealed electrode cavity 907. For example, when two lumens 910′ and 910″ are provided, one lumen can be used for fluid fill and the other lumen can be used for fluid withdrawal. In some embodiments, a continuous circulation of fluid can be provided through the sealed electrode cavity 907 using two lumens 910′ and 910″ during lithotripsy operation. In other embodiments, periodic circulation of fluid can be provided through the sealed electrode cavity 907 using two lumens 910′ and 910″ during lithotripsy operation. Continuous or periodic circulation of fluid through the sealed electrode cavity 907 during lithotripsy operation can remove heat introduced from the electrical discharges to control the temperature of the fluid in the electrode cavity and/or to replace the fluid as necessary if it breaks down from the electrical discharges. Further, continuous or periodic circulation of fluid through the sealed electrode cavity 907 during lithotripsy operation can remove lingering air or gas bubbles from the cavity that can otherwise interfere with the transmission of the lithotripsy shock waves or disrupt optimal therapeutic effect. In some embodiments, when lumens 910 or 910′ and 910″ are provided, the respective lumens can be configured with high fluid inductance to prevent significant fluid backflow from the shock waves produced in the electrode cavity 907. In other embodiments, when lumens 910 or 910′ and 910″ are provided, a flow control unit can be provided to allow flow through the respective lumens to occur between shock waves and to stop flow in the lumens during shock waves. After the tunneling probe 904 forms the desired channel through, or partially through, the calcified material, the tunneling catheter 900 can be withdrawn and dilation of the occlusion can be performed by a separate dilation balloon.

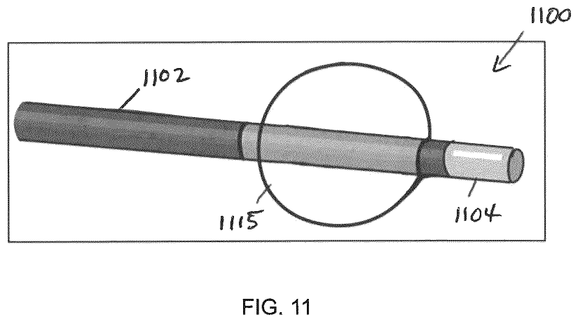

Referring now to FIG. 11, there is illustrated another lithotripsy tunneling catheter in accordance with a tenth aspect. The tunneling catheter 1100 includes a catheter shaft 1102 and tunneling probe 1104 on the distal end. The tunneling probe 1104 can be configured as, but is not limited to, any of the configurations previously described for probes 104, 204, 304, 404, 504, 604, 704, 804 and 904. The tunneling catheter 1100 further includes a positioning balloon 1115 attached to the catheter shaft behind (i.e., proximal to) the tunning probe 1104. The positioning balloon 1115 can be inflated with an inflation fluid (e.g., saline) via an inflation lumen (not shown) in the catheter shaft 1102. In the illustrated embodiments, the positioning balloon 1115 is configured to extend equally around the catheter shaft 1102 to center the tunneling probe 1104 in the vessel lumen. In other embodiments, the positioning balloon 1115 can be configured to extend unequally around the catheter shaft 1102 to offset the tunneling probe 1104 from the centerline of the vessel lumen.

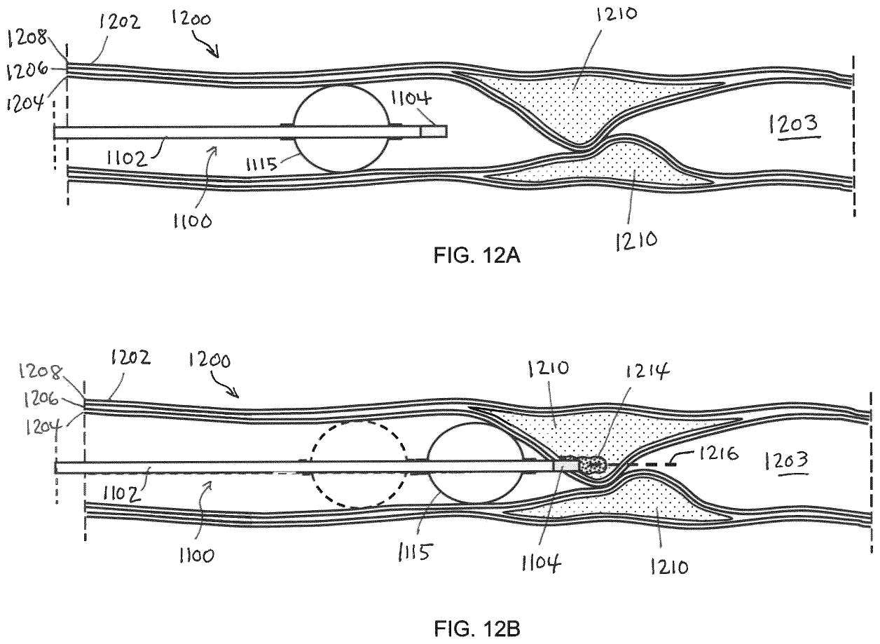

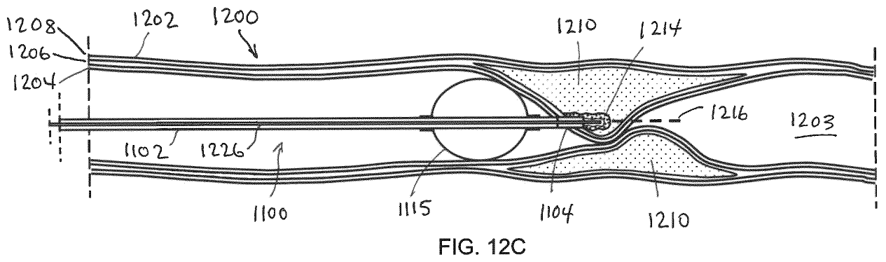

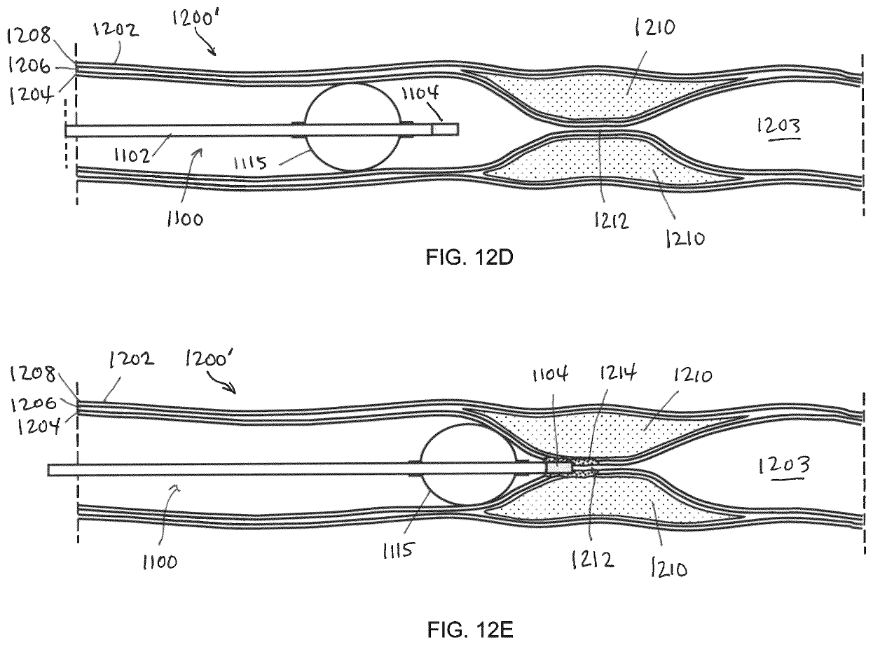

Referring now to FIGS. 12A, 12B and 12C, use of the tunneling catheter 1100 within an artery or blood vessel that is totally occluded by calcified tissue is illustrated. The vessel 1200 comprises a vessel wall 1202 defining interior lumen 1203 filled with fluid (e.g., blood). The vessel wall 1202 comprises multiple layers including intima 1204, media 1206 and externa 1208. Areas of calcified plaques 1210 within the vessel wall 1202 form occlusions that reduce or block the flow of blood through the vessel. In the illustrated embodiment of FIGS. 12A-12C, the vessel is completely blocked.

Referring specifically to FIG. 12A, the tunneling catheter 1100 is shown disposed within the vessel 1200 near the occlusion 1210. The positioning balloon 1115 can be inflated to center the tunneling probe 1104 within the vessel lumen 1203. The tunneling catheter 1100 can be advanced through the vessel lumen 1203 until the tunneling probe 1104 is adjacent the calcified plaques 1210. By activating the pulse generator, electrical discharges are produced between the electrodes of the tunneling probe 1104 to send hydraulic shock waves into the adjacent calcified material 1210. These shock waves crack, pulverize or otherwise break down the nearby calcified material of the plaque 1210, transforming it into a weakened material or “modified plaque.” The modified plaque has less strength and/or cohesion than the original calcified material, and thus has less resistance to mechanical dislocation (i.e., being pushed aside or pushed through) than the original calcified material. Referring now also to FIG. 12B, after operation of the tunneling probe 1104, portions of the calcified plaque 1210 within effective range of the tunneling probe are broken down by the shock waves into a “pocket” of modified plaque 1214. The modified plaque 1214 can then be penetrated or pushed aside (i.e., dislocated) to form a channel through the calcified plaque 1210 using substantially less force than would be required to penetrate or push aside the original calcified plaques.

Referring now also to FIG. 12C, in some cases, the tunneling probe 1104 itself can be used to push thorough or dislodge the modified plaque 1214 to form the channel in the calcified tissue. In some embodiments, e.g., wherein the tunneling probe 1104 includes a guidewire lumen (e.g., probes 204, 604, 704 and 804), a guidewire 1226 can be introduced through the catheter shaft 1102 and tunneling probe to channel through the modified plaques as shown in FIG. 12C. In other cases, the tunneling probe 1104 may be withdrawn from the vessel and a guidewire 1226 or other tool separately inserted to channel through the modified plaque.

Referring now to FIGS. 12D and 12E, use of the tunneling catheter 1100 within an artery or blood vessel having a lumen with a portion substantially narrowed (i.e., but not totally blocked) by calcified tissue is illustrated. Referring first to FIG. 12D, the tunneling catheter 1100 is shown disposed within the vessel 1200′ near the occlusion formed by calcified tissue 1210. The positioning balloon 1115 can be inflated to position the tunneling probe 1104 at the desired position within the vessel lumen 1203 such that the tunneling probe 1104 is adjacent the narrowed lumen portion 1212. Upon activating the pulse generator, electrical discharges are produced between the electrodes of the tunneling probe 1104 to send hydraulic shock waves into the adjacent calcified material 1210 along the narrowed lumen portion 1212. These shock waves break down the nearby calcified material of the plaque 1210, transforming it into modified plaque. Referring now also to FIG. 12E, after operation of the tunneling probe 1104, portions of the calcified plaque 1210 along the narrowed lumen 1212 are broken down by the shock waves into a “pocket” of modified plaque 1214. The modified plaque 1214 can then be penetrated or pushed aside (i.e., dislocated) to form a channel through the calcified plaque 1210 using substantially less force than would be required to penetrate or push aside the original calcified plaques.

After advancing the tunneling catheter 1100 to position the tunneling probe 1104 in the newly formed channel through the modified plaques 1214 along the narrowed portion 1212 of the lumen, the pulse generator can be activated once again so that the tunneling probe 1104 can break down further calcified tissue 1210 into further modified plaques. These newly modified plaques 1214 can then be channeled as previously described. This process is repeated to incrementally extend the channel until it crosses the occlusion. In many cases, the activated tunneling probe 1104 will naturally follow the path of least resistance along the narrowed portion 1212 of the lumen as it advances through the calcified tissues 1210 to form the channel.

In some cases, the tunneling catheter 1100 can include a suction and/or an irrigation lumen, in which case the tunneling process can include using irrigation fluid to loosen the modified plaque 1414 and/or using suction to remove the pulverized or modified plaque. When necessary, the tunneling process can include alternately shocking the calcified plaques 1210 using the tunneling probe 1104 and channeling the modified plaque 1214 multiple times until a channel is formed through, or partially through, the occlusion 1212 (indicated by dashed line 1216). After a suitable channel is formed in the calcified plaques 1210 of the occlusion 1212, the tunneling catheter 1100 can be withdrawn and dilation of the occlusion can be performed by a separate dilation balloon.

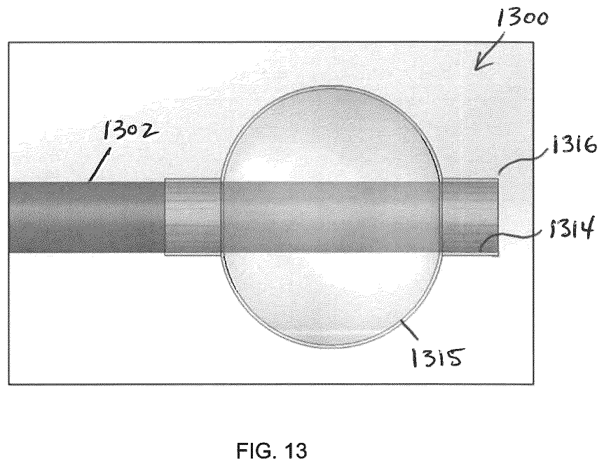

Referring now to FIG. 13, there is illustrated a centering support catheter in accordance with another aspect for use in connection with a lithotripsy tunneling catheter including, but not limited to, tunneling catheters 100, 200, 300, 400, 500, 600, 700, 800 and 900. The support catheter 1300 includes a catheter shaft 1302 defining an internal lumen 1314 and a positioning balloon 1315 disposed adjacent the support catheter's distal end 1316. The positioning balloon 1315 can be inflated with an inflation fluid (e.g., saline) via an inflation lumen (not shown) in the catheter shaft 1302. In the illustrated embodiments, the positioning balloon 1315 is configured to extend equally around the catheter shaft 1302 to center distal end 1316 in a vessel lumen. In other embodiments, the positioning balloon 1315 can be configured to extend unequally around the catheter shaft 1302 to offset the distal end 1316 from the centerline of the vessel lumen. The support catheter 1300 does not include a tunneling probe, but rather is configured to receive a respective lithotripsy tunneling catheter into the internal lumen 1314 and guide the tunneling catheter through the lumen until its tunneling probe extends from the distal end 1316.

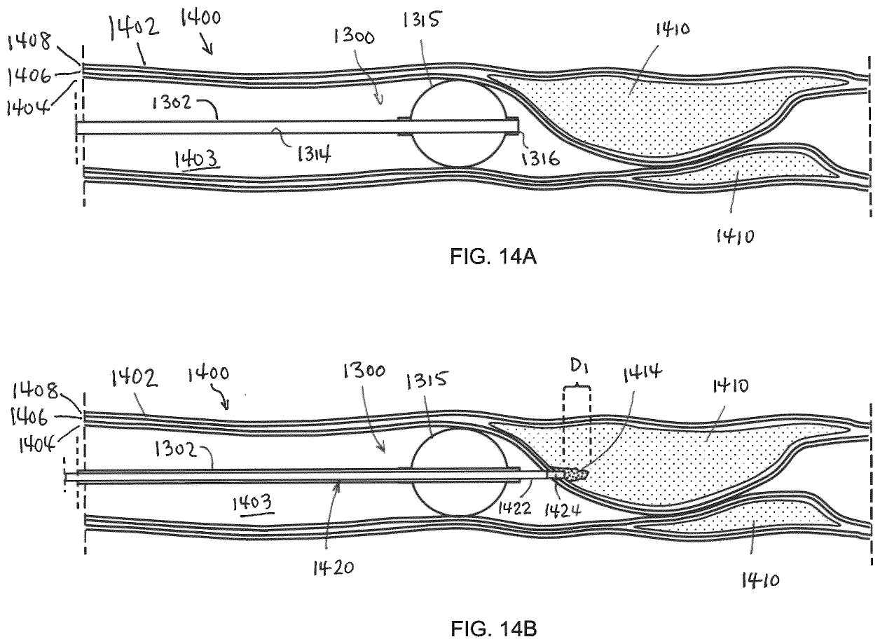

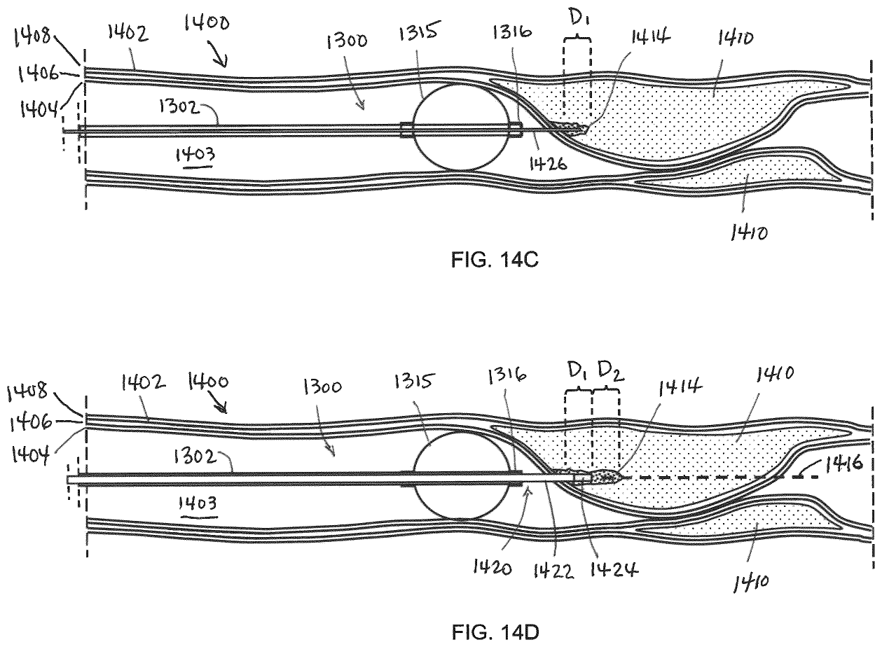

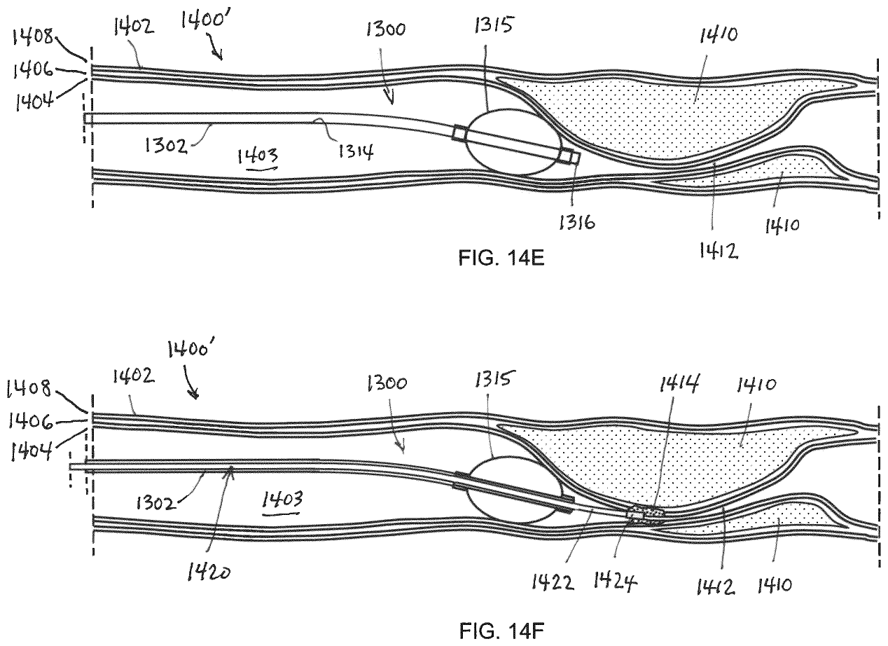

Referring now to FIGS. 14A, 14B, 14C and 14D, use of the support catheter 1300 and lithotripsy tunneling catheter 1420 within an artery or blood vessel that is totally occluded by calcified material is illustrated. The vessel 1400 comprises a vessel wall 1402 defining interior lumen 1403 filled with fluid (e.g., blood). The vessel wall 1402 comprises multiple layers including intima 1404, media 1406 and externa 1408. Areas of calcified plaques 1410 within the vessel wall 1402 form occlusions 1412 that reduce or block the flow of blood through the vessel. In the illustrated embodiment of FIGS. 14A-14D, the vessel is completely blocked. The tunneling catheter 1420 can be configured as, but is not limited to, any of the configurations previously described for tunneling catheters 100, 200, 300, 400, 500, 600, 700, 800 and 900 including a tunneling catheter shaft 1422 having a lithotripsy tunneling probe 1424 on the distal end. The lithotripsy tunneling probe 1424 can be configured as, but is not limited to, any of the configurations previously described for tunneling probes 104, 204, 304, 404, 504, 604, 704, 804 and 904.

Referring now specifically to FIG. 14A, the support catheter 1300 can be introduced into the vessel 1400 with or without the use of a guidewire. Preferably, the positioning balloon 1315 is uninflated during introduction to facilitate travel through the intervening lumens of the body. Once the support catheter 1300 is positioned at a desired location near the occlusion 1412, the positioning balloon 1315 can be inflated to center or otherwise position the distal end 1316 of the support catheter within the vessel 1400. The support catheter 1300 can optionally be secured in place (e.g., at the introduction point) to prevent its unintended movement within the vessel 1400 during subsequent operations. Once the support catheter 1300 is located at the desired position, the guidewire (if used) can be withdrawn.

Referring now also to FIG. 14B, the tunneling catheter 1420 can be introduced through the lumen 1314 of the support catheter 1300 and advanced through the lumen by pushing the catheter shaft 1422 until the tunneling probe 1424 extends from the distal end 1316 to a desired location adjacent the calcified plaques 1410 of the occlusion 1412. By activating the pulse generator, electrical discharges are produced between the electrodes of the tunneling probe 1424 to emit hydraulic shock waves into the adjacent calcified material 1410. These shock waves break down portions of the calcified material 1410 within the effective range of the tunneling probe 1424 into modified plaque 1414 as previously described. In the illustrated embodiment of FIG. 14B, the tunneling probe 1424 has an effective range (denoted “D1” and shown by dashed lines) extending primarily axially in front of the probe, creating a pocket of modified plaque 1414 having a depth “D1”. In other embodiments, the shock waves emitted from the tunneling probe 1424 can have different effective range and/or a different orientation, and thus create a pocket of modified plaque 1414 having different shape or dimensions. The modified plaque 1414 can be penetrated or pushed aside to form a channel through the calcified plaque 1410 using substantially less force than would be required to penetrate or push aside the original calcified plaques.

Referring now also to FIG. 14C, after operation of the tunneling probe 1424 to break up the calcified material 1410 into modified plaque 1414 to a depth of D1, a channel can be formed in the modified plaque by pushing it aside. In some cases, the tunneling probe 1424 itself can be used to push through or dislodge the newly modified plaque 1414 to form the channel. In other cases, a guidewire can be used for channeling through the modified plaques. For example, in embodiments having a tunneling probe 1424 that includes a guidewire lumen (e.g., probes 204, 604, 704 and 804), the tunneling catheter 1420 can remain positioned in the support catheter 1300 and a guidewire 1426 can be introduced through the tunneling catheter shaft 1402 and tunneling probe to channel through the modified plaques. In other embodiments having a tunneling probe 1424 does not includes a guidewire lumen (e.g., probes 104, 204, 404, 504 and 904), the tunneling catheter 1420 can be withdrawn from the support catheter 1300 and the guidewire 1426 can be introduced through the support shaft 1302 to channel through the modified plaques. In the illustrated embodiment of FIG. 14C, the tunneling catheter 1420 has been temporarily withdrawn from the support catheter 1300 so that a separate guidewire 1426 or other tool can be inserted back to the occlusion site 1412 and used to channel through the modified plaque 1414 to a depth of D1. Use of a guidewire 1426 for channeling can avoid the chance of damaging the tunneling probe 1424 during channelization of the calcified material 1410. In other embodiments, the tunneling catheter 1420 can be temporarily withdrawn from the support catheter 1300 so that a tool catheter having irrigation and/or suction lumens can be inserted back to the occlusion site 1412 and used to channel through the modified plaque 1414 using irrigation and/or suction.

Referring now also to FIG. 14D, after making the initial channelization to a depth D1, the tunneling catheter 1420 can be reinserted (if withdrawn) through the support catheter 1300 to the occlusion site 1412 and advanced to the end of the channel. The tunneling process can then resume by reactivating the tunneling probe 1424 to shock the next interval, denoted D2, to break up additional calcified plaques 1410 into modified plaque 1414. After shocking the interval D2 with the tunneling probe 1424, the interval D2 can be channelized as previously described. The process of first using shock waves to break up calcified tissues and then channelizing through the modified plaque is repeated until a channel of desired length is formed through the occlusion 1412 are (indicated by dashed line 1416). In preferred embodiments, the support catheter 1300 acts as a “base of operations” that remains fixed in place during then entire tunneling process including during use of the tunneling probe 1424 and switching between the tunneling catheter 1420, guidewire 1426 and tool catheter (if used) to protect the walls 1402 of the vessels. After a suitable channel 1416 is formed in the calcified plaques 1410 of the occlusion 1412, the tunneling catheter 1420 can be withdrawn from the support catheter 1300 so that dilation of the occlusion can be performed by a separate dilation balloon. In some cases, the support catheter 1300 can be used to guide the separate dilation balloon to the occlusion location, whereas in other cases, the support catheter can be withdrawn before the dilation balloon is inserted.

Referring now to FIGS. 14E and 14F, use of the support catheter 1300 and the tunneling catheter 1420 within an artery or blood vessel having a lumen with a portion substantially narrowed (i.e., but not totally blocked) by calcified tissue is illustrated. Referring first to FIG. 14E, the support catheter 1300 is disposed within the vessel 1400′ near the occlusion formed by calcified tissue 1410. The positioning balloon 1315 can be inflated to position the support catheter 1300 at the desired position and orientation within the vessel lumen 1403 such that the distal end of the support catheter 1316 is adjacent the narrowed lumen portion 1412. In the illustrated embodiment of FIG. 14E, the positioning balloon 1315 has been selectively inflated and manipulated to allow the distal end 1316 of the support catheter 1300 to be positioned at an offset from the center of the lumen 1403. Referring now also to FIG. 14F, after positioning the support catheter 1300, the tunneling catheter 1420 can be introduced through the support catheter shaft 1302 and advanced until the tunneling probe 1424 extends from the support catheter to a position adjacent the narrowed lumen portion 1412 formed by the calcified tissue 1410. Upon activating the pulse generator, electrical discharges are produced between the electrodes of the tunneling probe 1104 to send hydraulic shock waves into the adjacent calcified material 1410 along the narrowed lumen portion 1412. These shock waves break down the nearby calcified material of the plaque 1410, transforming it into modified plaque. After operation of the tunneling probe 1104, portions of the calcified plaque 1410 along the narrowed lumen 1412 are broken down by the shock waves into a “pocket” of modified plaque 1414. The modified plaque 1414 can then be penetrated or pushed aside (i.e., dislocated) to form a channel through the calcified plaque 1410 using substantially less force than would be required to penetrate or push aside the original calcified plaques.

After advancing the tunneling catheter 1420 to position the tunneling probe 1424 in the newly formed channel through the modified plaques 1414 along the narrowed portion 1412 of the lumen, the pulse generator can be activated once again so that the tunneling probe 1424 can break down further calcified tissue 1410 into further modified plaques. These newly modified plaques 1414 can then be channeled as previously described. This process is repeated to incrementally extend the channel until it crosses the occlusion. In many cases, the activated tunneling probe 1424 will naturally follow the path of least resistance along the narrowed portion 1412 of the lumen as it advances through the calcified tissues 1410 to form the channel.

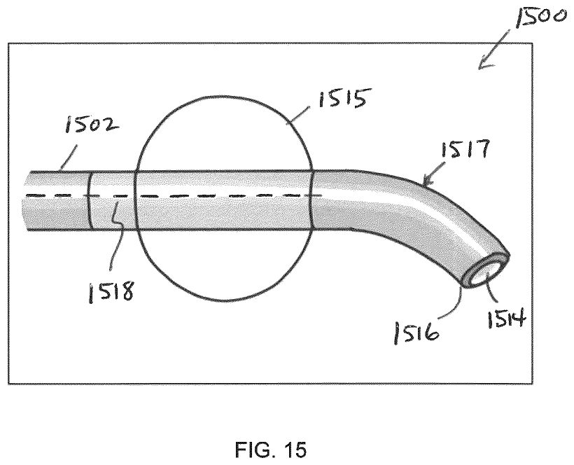

Referring now to FIG. 15, there is illustrated an alternative centering support catheter in accordance with another aspect for use in connection with a lithotripsy tunneling catheter including, but not limited to, tunneling catheters 100, 200, 300, 400, 500, 600, 700, 800 and 900. The alternative support catheter 1500 includes a catheter shaft 1502 defining an internal lumen 1514 and a positioning balloon 1515 disposed adjacent the support catheter's distal end 1516. The positioning balloon 1515 can be inflated with an inflation fluid via an inflation lumen (not shown) in the catheter shaft 1502. The distal end 1516 of the alternative support catheter 1500 further includes a guide tip 1517 that redirects the internal lumen 1514 in a predetermined manner. In the illustrated embodiment, the guide tip 1517 is curved at an angle of 34 degrees relative to the longitudinal axis 1518 of the support catheter shaft 1502. In other embodiments, the guide tip 1517 can be curved at an angle within the range from 20 degrees to 40 degrees relative to the longitudinal axis 1518 of the support catheter shaft 1502. In still other embodiments, the guide tip 1517 can be curved at an angle within the range from 10 degrees to 50 degrees relative to the longitudinal axis 1518 of the support catheter shaft 1502.

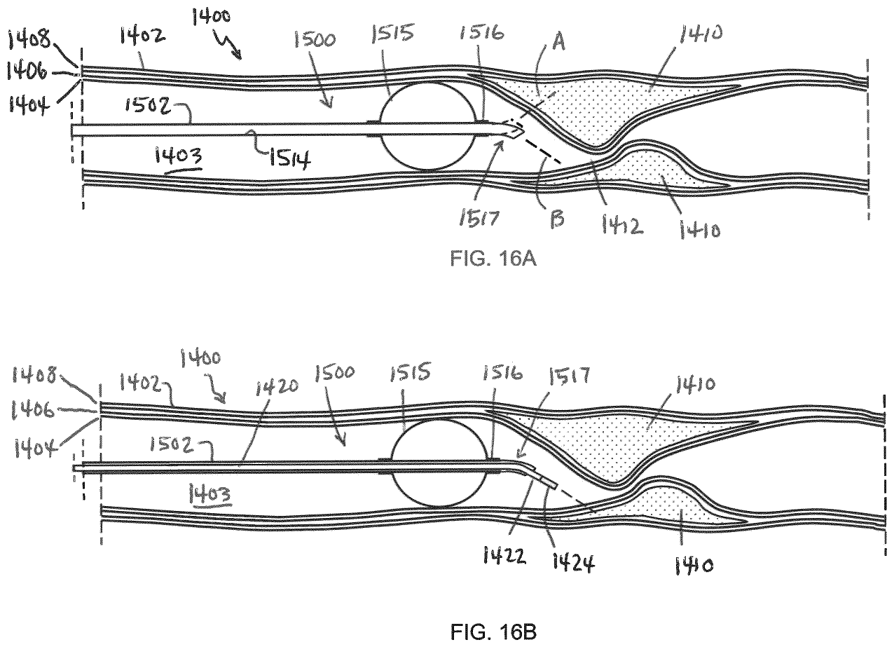

Referring now also to FIGS. 16A and 16B, use of the alternative support catheter 1500 and lithotripsy tunneling catheter 1420 within an occluded artery or blood vessel is illustrated. Unless otherwise indicated, the vessel 1400 and lithotripsy tunneling catheter 1420 are as previously described.

Referring first to FIG. 16A, the alternative support catheter 1500 can be introduced into the vessel 1400 with or without the use of a guidewire. Preferably, the positioning balloon 1515 is uninflated during introduction to facilitate travel through the intervening lumens of the body. The alternative support catheter 1500 is placed in the vessel 1400 such that the guide tip 1517 is directed in desired direction. Once the alternative support catheter 1500 is positioned at a desired location near the occlusion 1412, the positioning balloon 1515 can be inflated to center or otherwise position the distal end 1516 and guide tip 1517 within the vessel 1400. In some embodiments, the guide tip 1517 is made of radiopaque material or has radiopaque markings so that the exit direction of the guide tip can be imaged with radiography. In some embodiments, the direction of the guide tip 1517 can be selectively changed by twisting the catheter shaft 1502 prior to inflation of the positioning balloon 1515 as denoted by direction A and direction B in FIG. 16A. In some embodiments, the direction of the guide tip 1517 can also be selectively changed by twisting the catheter shaft 1502 after inflation of the positioning balloon 1515. Once the alternate support catheter 1500 is located at the desired position, the guidewire (if used) can be withdrawn.

Referring now also to FIG. 16B, the tunneling catheter 1420 can be introduced through the lumen 1514 of the support catheter 1500 and advanced through the lumen by pushing the catheter shaft 1422 until the tunneling probe 1424 extends from the guide tip 1517 at the distal end 1516. Since the guide tip 1517 is curved at an angle relative to the longitudinal axis 1518 of the support catheter shaft 1502, the distal end of the tunneling catheter 1420 including the tunneling probe 1424 will exit the guide tip at a similar angle relative and in a similar direction. The tunneling probe 1424 can then be advanced from the guide tip 1517 to a desired location adjacent the calcified plaques 1410 of the occlusion 1412. By activating the pulse generator, electrical discharges are produced between the electrodes of the tunneling probe 1424 to emit hydraulic shock waves into the adjacent calcified material 1410 as previously described, except now the shock waves can be directed at selected angles and at locations not centered in the vessel 1400. The use of a tunneling catheter 1420 with the alternative support catheter 1500 is otherwise substantially similar to the use of support catheter 1300 as previously described.

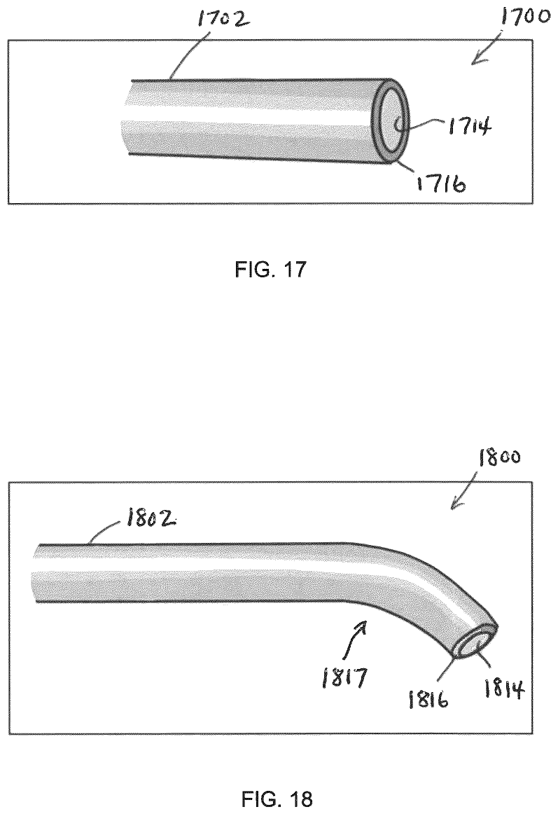

Referring now to FIG. 17, there is illustrated yet another support catheter in accordance with another aspect for use in connection with a lithotripsy tunneling catheter 1420. The straight support catheter 1700 includes a catheter shaft 1702 defining an internal lumen 1714 as previously described but does not include a positioning balloon. After positioning the distal end 1714 of the catheter shaft 1702 at a desired location adjacent an occlusion 1412, the proximal end (not shown) can be secured at the introducing site such that the distal end remains fixed relative to the occlusion. A tunneling catheter 1420 can then be introduced through the straight support catheter 1700 until the tunneling probe 1424 is adjacent to the calcified tissue 1410. The straight support catheter 1700 can therefore act as a “base of operations” that remains fixed in place during then entire tunneling process including during use of the tunneling probe 1424 and switching between the tunneling catheter 1420, guidewire 1426 and tool catheter (if used) to protect the walls 1402 of the vessels. After a suitable channel 1416 is formed in the calcified plaques 1410 of the occlusion 1412, the tunneling catheter 1420 can be withdrawn from the straight support catheter 1700 so that dilation of the occlusion can be performed by a separate dilation balloon. The straight support catheter 1700 does not provide the centering features of the support catheter 1300, but it is simpler to operate and creates fewer steps in workflow. The straight support catheter 1700 is therefore well suited for use in smaller vessels or where additional centering of the tunneling probe is not required.

Referring now to FIG. 18, there is illustrated still another support catheter in accordance with another aspect for use in connection with a lithotripsy tunneling catheter 1420. The curved support catheter 1800 includes a catheter shaft 1802 defining an internal lumen 1814 as previously described but does not include a positioning balloon. The curved support catheter 1800 further includes a curved guide tip 1817 on its distal end 1816 similar to the guide tip 1517 of the alternative support catheter 1500. The curved support catheter 1800 be used to direct a tunneling catheter 1420 with tunneling probe 1424 to a desired location at a desired angle substantially as described for the alternative support catheter 1500, but not including the centering features provided by the centering balloon 1515 of the alternative support catheter. The curved support catheter 1800 can therefore act as a “base of operations” that remains fixed in place during then entire tunneling process including during use of the tunneling probe 1424 and switching between the tunneling catheter 1420, guidewire 1426 and tool catheter (if used) to protect the walls 1402 of the vessels. After a suitable channel 1416 is formed in the calcified plaques 1410 of the occlusion 1412, the tunneling catheter 1420 can be withdrawn from the curved support catheter 1800 so that dilation of the occlusion can be performed by a separate dilation balloon. While the straight support catheter 1700 does not provide the centering features of the support catheter 1300, it is simpler to operate and creates fewer steps in workflow.

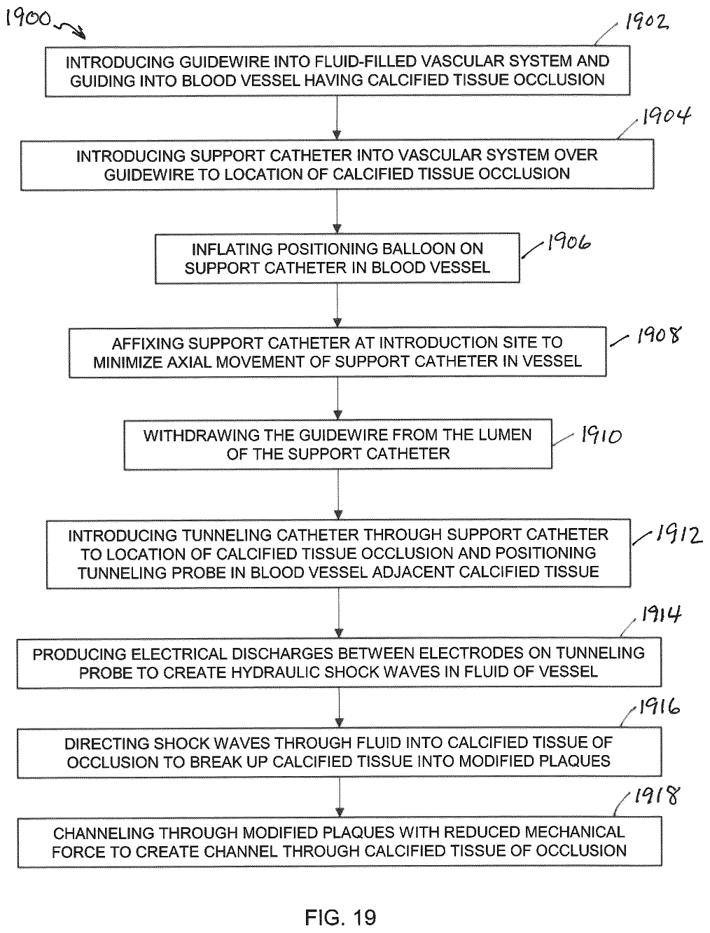

Referring now to FIG. 19, there is illustrated a method for using a lithotripsy device for tunneling a crossing through vascular occlusions formed of calcified tissue. The method 1900 includes providing a lithotripsy tunneling catheter 1420 and a support catheter 1300 as previously described. At block 1902 is the step of introducing a guidewire 1426 into a fluid-filled vascular system and guiding the guidewire into a blood vessel 1400 having a calcified tissue occlusion 1410, 1412. At block 1904 is the step of introducing the support catheter 1300 into the vascular system 1400 over the guidewire 1426 to the location of the calcified tissue occlusion 1410, 1412. At block 1906 is the step of inflating a positioning balloon 1315 on the support catheter 1300 in the blood vessel 1400. At block 1908 is the step of affixing the support catheter 1300 at introduction site to minimize axial movement of support catheter in the vessel 1400. At block 1910 is the step of withdrawing the guidewire 1426 from the lumen 1314 of the support catheter 1300. At block 1912 is the step of introducing the tunneling catheter 1420 through support catheter 1300 to location of calcified tissue occlusion 1412 and positioning the tunneling probe 1424 in the blood vessel 1400 adjacent the calcified tissue 1410. At block 1914 is the step of producing electrical discharges between the electrodes, e.g., 706, 708, on the tunneling probe 1424 to create hydraulic shock waves in the fluid of the vessel 1400. At block 1916 is the step of directing the shock waves produced by the electrical discharges through the fluid in the vessel 1400 into the calcified tissue 1410 of occlusion 1412 to break up the calcified tissue 1414. At block 1918 is the step of channeling through the modified plaque 1414 with reduced mechanical force to create a channel through the calcified tissue 1410 of the occlusion 1412 along a desired line 1416.

The method 1900 is subject to revisions to the order and detail of the steps in accordance with the previous disclosure. For example, the steps 1916 of breaking up the calcified tissue 1410 into modified plaque and 1918 of channeling through the modified plaque 1414 with reduced force to create a channel through the calcified tissue may be repeated as necessary. Further, the method 1900 is subject to the addition and deletion of steps in accordance with the previous disclosure. For example, in some embodiments, the step 1916 of breaking up the calcified tissue 1410 into modified plaque 1414 can be followed by a new step of withdrawing the tunneling catheter 1420 from the support catheter 1300 and inserting a guidewire 1426 to perform the step 1918 of channeling though the modified plaque. Many other methods for using a lithotripsy device for tunneling a crossing through vascular occlusions formed of calcified tissue are disclosed by examination of the previous disclosure.

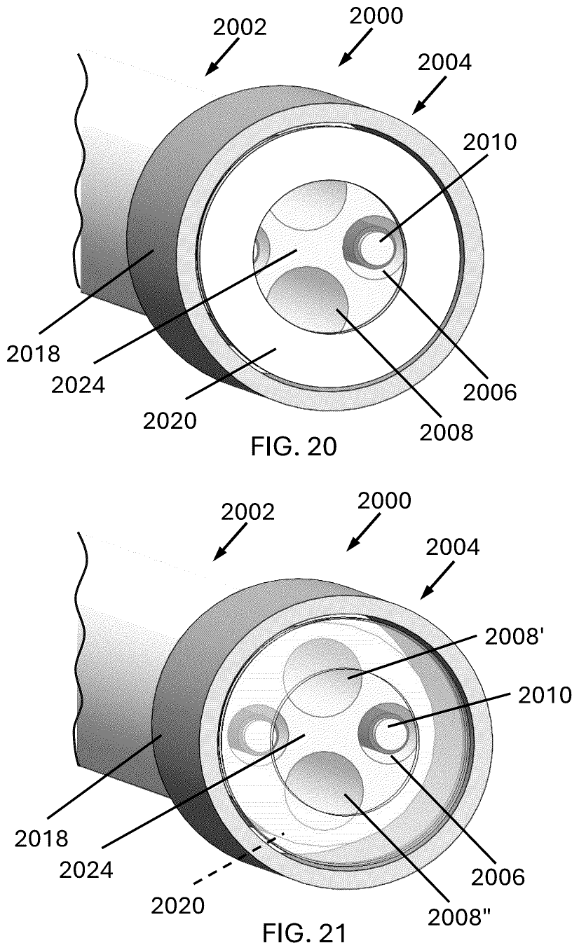

Referring now to FIGS. 20-22, there is illustrated a lithotripsy catheter with a sealed-chamber “drum” type tunneling probe in accordance with another embodiment. Unless otherwise described, this embodiment operates in a fashion similar to the embodiment previously described in connection with FIGS. 9-10. The tunneling lithotripsy catheter 2000 includes a multi-lumen catheter shaft 2002 and tunneling probe (or “end effector”) 2004 affixed on the distal end of the catheter shaft.

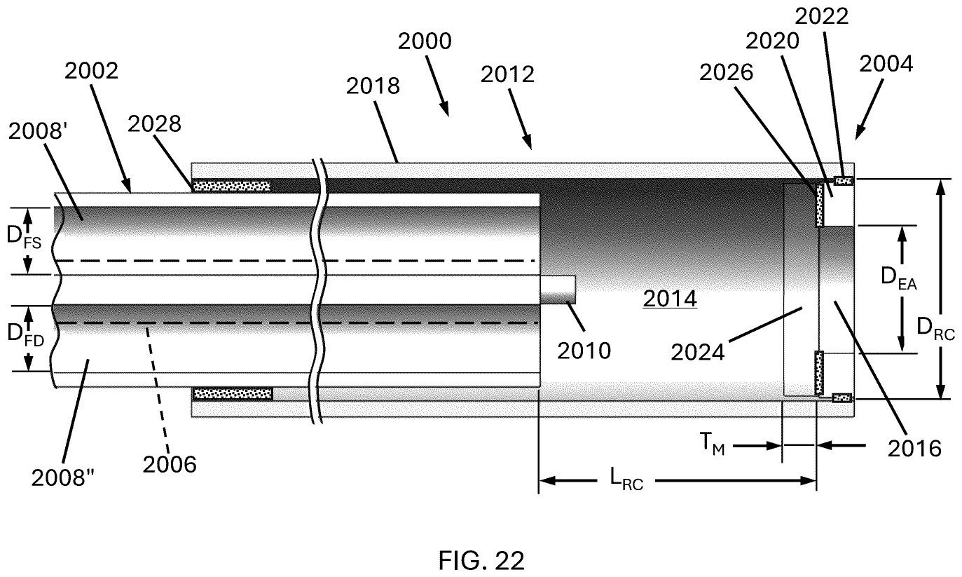

As best seen in FIGS. 21 and 22, the multi-lumen catheter shaft 2002 defines one or more electrode lumens 2006 and one or more fluid lumens 2008 extending therethrough from its proximal end to its distal end. Electrodes 2010 extend from the distal end of the catheter shaft 2002 and can be electrically connected to an external electrical power supply (not shown) via electrical leads (not shown) running through the electrode lumens 2006. In the illustrated embodiment, two electrodes 2010, two electrode lumens 2006 and two fluid lumens 2008 are provided; however, in other embodiments the catheter shaft 2002 may provide a different number of electrode lumens and/or fluid lumens. In the illustrated embodiment, a first fluid lumen 2008′ can be a supply fluid lumen and a second fluid lumen 2008″ can be a drain fluid lumen. The fluid lumens 2008 can be fluidly connected to an external fluid pump system (not shown) to circulate a fluid, e.g., saline, through the resonating chamber of the end effector 2004. The multi-lumen catheter shaft 2002 can be made of Pebax® (a trademark of Arkema) brand thermoplastic elastomer, other thermoplastic elastomers, or other polymers or materials used in conventional catheter shafts. The supply and drain fluid lumens 2008′ and 2008″ should have diameters selected to provide a manageable pressure drop in the working fluid circulating through the catheter lumens to keep the circulating pump pressure at a manageable level and to avoid excessive static pressure in the resonating chamber 2014 that could cause failure of the flexible membrane 2024. Other variables can require an optimization equation. The lumens 2008 need to be large enough to reduce pump pressure for the optimal fluid flow rate for cooling the electrodes 2010 and evacuating air bubbles from the resonating chamber 2014. Lumens 2008 that are too large may introduce more air bubbles. Thus, the fluid lumens 2008 should have diameters selected to provide manageable pressure drop, facilitate active cooling of the electrodes 2010, and evacuate air bubbles created by electrical discharges and subsequent cavitation. In the illustrated embodiment, the supply and drain fluid lumens 2008′ and 2008″ each have respective diameters R_{FS} and R_{FD} of about 0.5 mm.

The tunneling probe 2004 of the lithotripsy catheter assembly 2000 further comprises a rigid outer shell or “can” 2012 defining a resonating chamber 2014 therewithin and an emitting aperture 2016. The emitting aperture 2016 typically opens in the distal direction along the central (longitudinal) axis of the catheter shaft. In the illustrated embodiment, the can 2012 includes a rigid tube portion 2018 and a rigid annular retainer ring 2020 affixed perpendicularly across the distal end of the tube portion. The center opening of the retaining ring 2020 defines the emitting aperture 2016. In the illustrated embodiment, the tube portion 2108 can be formed from a stainless steel tube, for example a hypo tube, and the retaining ring 2020 can be made from a stainless steel ring, for example a flat washer. In other embodiments, the tube portion 2018 and/or the retaining ring 2020 can be formed of other rigid materials. In the illustrated embodiment, a first bond 2022 between the retaining ring 2020 and the tube portion 2018 is made using laser welding; however, in other embodiments the first bond can be made using other forms of welding, adhesives or other known bonding or joining methods. In still other embodiments, the retaining ring 2020 can be integrally formed from the tube portion 2018 by spin forming a portion of the sidewall (of the tube portion) from an axial orientation to a radial orientation (i.e., relative to the central axis).

Referring still to FIGS. 20-22, a flexible membrane 2024 is attached to the can 2012 to cover the emitting aperture 2016. The membrane 2024 must be elastic to provide good transmission of pressure pulses created in the resonating chamber 2014, but at the same time it must be strong enough to resist rupturing. Generally speaking, the softer the material of the flexible membrane 2024, the better the pressure pulse transmission from the resonating chamber 2014 to the exterior medium having the target tissue. In some embodiments, the membrane 2024 is formed of an elastomer material having a durometer within the range from 50 Shore A to 35 Shore D. In some preferred embodiments, the membrane 2024 is formed of an elastomer material having a durometer within the range from 80 Shore A to 90 Shore A. In some embodiments, the elastomer material of the membrane 2024 is a polyurethane material, whereas in other embodiments, other elastomer or polymer materials can be used. In still other embodiments, the membrane 2024 can be made from a thin metallic diaphragm or other non-polymeric material, provided the membrane is sufficient flexibility in the axial direction to transmit acoustic pressure waves.

As best seen in FIG. 22, in the illustrated embodiment, the membrane 2024 is attached to the proximal face of the retaining ring 2020 with a second bond 2026, which is preferably fluid-tight and mechanically strong. In this illustrated embodiment, the second bond 2026 is formed using thermal bonding; however, in other embodiments the second bond can be made using adhesives or other known bonding or joining methods. The distal end of the catheter shaft 2002 is inserted into the proximal end of the tube portion 2018, and a third bond 2028 is formed therebetween to affix the can 2012 in position on the catheter shaft. This third bond 2028 is typically formed after the flexible membrane 2024 has been affixed to the can 2012 with the second bond 2026. In the illustrated embodiment, the third bond 2028 is a thermal or adhesive bond; however, in other embodiments the third bond can be formed using welding or other known bonding or joining methods. The third bond 2028 must be sufficient to provide a fluid seal and a mechanical coupling between the catheter shaft 2002 and the can 2012; however, it need not extend along the entire longitudinal interface between the shaft 2002 and the can 2012.

When the flexible membrane 2024 is affixed to the can 2012, and the can is affixed over the multi-lumen shaft 2002, a resonating chamber 2014 is defined between the distal end of the shaft, the walls of the can and the membrane. The can 2012 further defines an emitter aperture 2016 which is covered by the flexible membrane 2024 to seal the resonating chamber from the exterior environment.

During operation, the resonating chamber 2014 is filled with a working fluid (e.g., saline or saline-contrast mixture) that circulates through the multi-lumen shaft 2002 to evacuate air bubbles and cool the end effector components. Energy delivered by the generator to the exposed ends of the electrical leads (i.e., electrodes 2010) creates electrical discharges though the working fluid and subsequent fluid cavitation within the working fluid. Hydraulic shock waves form from this cavitation and move through the working fluid in the resonating chamber, being focused axially forward (i.e., in the distal direction) by the relatively rigid tube portion 2018 (having relatively high acoustic impedance) through the flexible membrane 2024 (having relatively low acoustic impedance) and into the external medium where they subsequently propagate into the target calcified tissue. Repeated exposure of the calcified tissue to hydraulic shock waves creates modified plaque that is more susceptible to dislocation than the original calcified tissue.

The current design parameters for the lithotripsy catheter 2000 are currently as follows. The internal diameter D_{RC} of the resonating chamber 2014 is influenced by the size of the expected body structures that must be transited to access the treatment location. For some peripheral vascular use and/or coronary vascular use, an overall (exterior) diameter for the catheter 2000 of 2.5 mm or less is preferred. Therefore, the internal diameter D_{RC} of the resonating chamber 2014 in some embodiments has a value within the range from 1.00 mm to 2.00 mm. The internal diameter D_{RC} in some preferred embodiments has a value within the range from 1.50 mm to 1.70 mm. The internal diameter D_{RC} in some current preferred embodiments has a value of about 1.60 mm. For other uses, the allowable exterior diameter of the catheter 2000 can be greater, and thus the internal diameter D_{RC} can be proportionally larger.

The diameter D_{EA} of the emitter aperture 2016 should be as large as possible to reduce the attenuation of the hydraulic pulses (i.e., acoustic pressure waves) as they are transmitted through the flexible membrane 2024. Thus, ideally, the ratio of emitter aperture diameter to resonating chamber diameter should approach 1.0 (i.e., the value of D_{EA}/D_{RC}→1.0). However, since there must be sufficient area on the exposed proximal face of the retainer ring 2020 to accommodate the second bond 2026 holding the membrane 2024 during use, the value of D_{EA}/D_{RC} of this design must be smaller than 1.0. For embodiments of catheter 2000 having an internal diameter D_{RC} within the range from 1.0 mm to 2.0 mm, the ratio D_{EA}/D_{RC} can have a value within the range of 0.4 to 0.8. For some preferred embodiments of catheter 2000 having an internal diameter D_{RC} within the range from 1.0 mm to 2.0 mm, the ratio D_{EA}/D_{RC} can have a value within the range of 0.5 to 0.6. In some current preferred embodiments having the chamber diameter D_{RC} of about 1.6 mm, the D_{EA} is about 0.9 mm.

The flexible membrane 2024 of the catheter 2000 should be as thin (i.e., in the axial direction) as possible to reduce attenuation of the hydraulic pulses/acoustic pressure waves transmitted across the membrane. Nevertheless, the membrane 2024 must be thick enough to resist failure during use. For embodiments of catheter 2000 having an internal diameter D_{RC} within the range from 1.0 mm to 2.0 mm, the thickness T_{M} of the membrane 2024 can have a value within the range of 0.050 mm to 0.254 mm. For some preferred embodiments of catheter 2000 having an internal diameter D_{RC} within the range from 1.0 mm to 2.0 mm, the thickness T_{M} can have a value within the range of 0.127 mm to 0.152 mm.

The length L_{RC} of the resonating chamber 2014 should be selected on the basis of multiple factors. First, as the value of length L_{RC} increases, there is more distance for the relatively violent/sharp pressure waves generated at the electrodes 2010 to dampen or attenuate before they reach the relatively fragile flexible membrane 2024. Thus, a relatively higher value for L_{RC} extends the life (i.e., reliability) of the flexible member 2024. However, if the value of length L_{RC} is too high, this can cause excessive damping/attenuation of the acoustic pressure waves before they reach the flexible member 2024, resulting in reduced efficiency and/or effectiveness of the acoustic waves transmitted by the catheter 2000 to the target calcifications. For embodiments of catheter 2000 having an internal diameter D_{RC} within the range from 1.0 mm to 2.0 mm, acceptable membrane life (or reliability) and acoustic efficiency can be achieved with a resonating chamber length L_{RC} having a value within the range of 1.0 mm to 6.0 mm. For some preferred embodiments of catheter 2000 having an internal diameter D_{RC} within the range from 2.0 mm to 4.0 mm, the resonating chamber length L_{RC} has a value within the range of 2.0 mm to 4.0 mm. In the illustrated embodiment, the length L_{RC} is measured in the axial (i.e., longitudinal) direction from the distal face of the catheter shaft 2002 to the distal face of the membrane 2024 for ease of measurement. In other embodiments, the length L_{RC} is measured in the axial direction from the distal face of the catheter shaft 2002 to the proximal face of the membrane 2024. When the thickness T_{M} of the membrane 2024 is small relative to L_{RC}, the choice of measurement surface has a reduced effect.

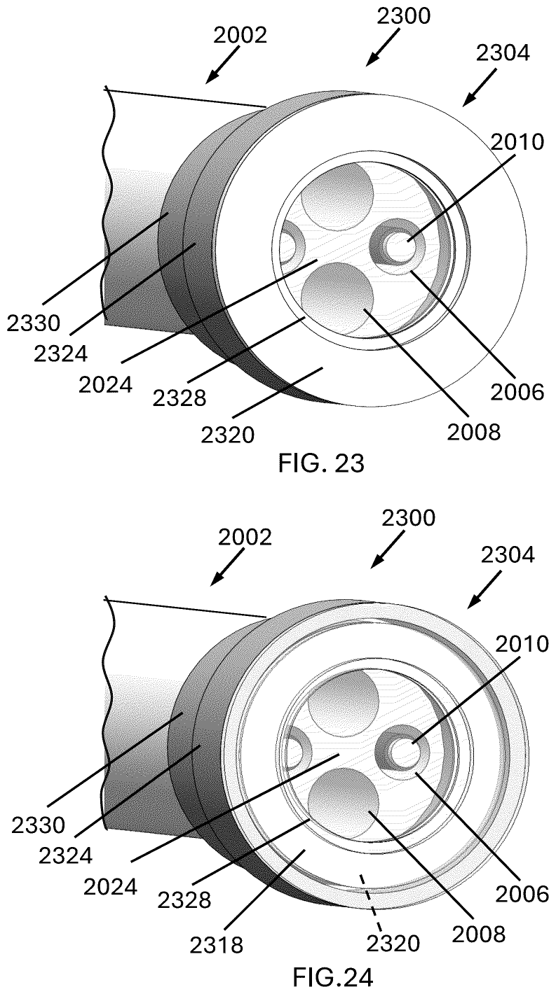

Referring now to FIGS. 23-25, there is illustrated a lithotripsy catheter with a sealed-chamber “drum” type tunneling probe in accordance with another embodiment. Unless otherwise described, this embodiment operates in a fashion similar to the embodiment previously described in connection with FIGS. 20-22. Elements in the description and drawings identified by the same reference numerals are identical or functionally equivalent to those previously described. The tunneling lithotripsy catheter 2300 includes a multi-lumen catheter shaft 2002 and tunneling probe/end effector 2304 affixed on the distal end of the catheter shaft. The end effector 2304 of this embodiment has a different structure for securing the flexible membrane 2024.

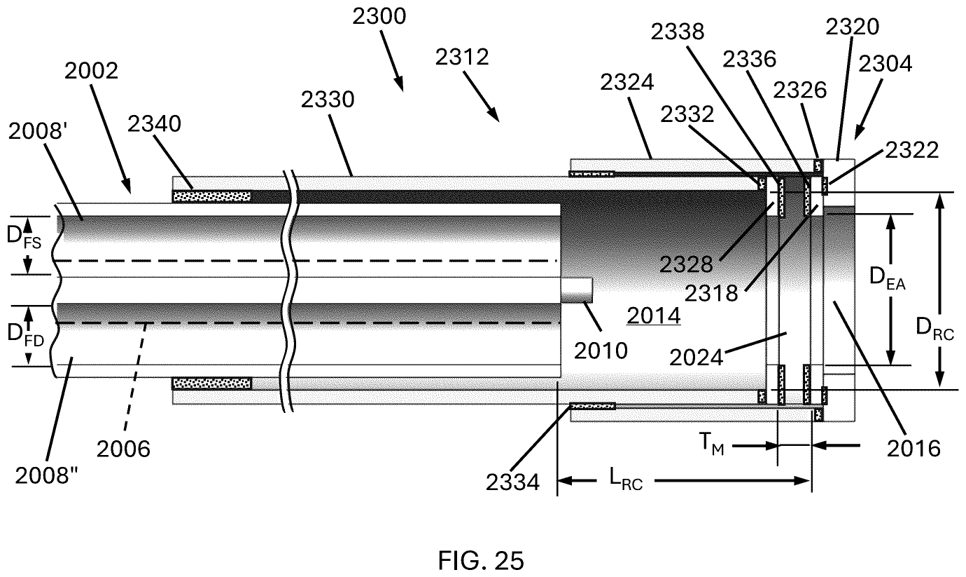

As best seen in FIGS. 24 and 25, the multi-lumen catheter shaft 2002 of this embodiment and its constituent elements including electrode lumens 2006, fluid lumens 2008, and electrodes 2010 are substantially similar to those previously described. The function and operation of the catheter shaft 2002 to produce electrically induced pressure waves is also similar. The tunneling probe 2304 comprises a rigid outer shell or “can” 2312 defining a resonating chamber 2014 therewithin and an emitting aperture 2016 open in the distal direction along the central axis of the catheter shaft. The can 2312 includes a rigid, annular outer retainer ring 2318 that is centered on a proximal face of a rigid, annular face ring 2320 and affixed with a first bond 2322. The face ring 2320 is affixed perpendicularly across the distal end of a rigid outer tube portion 2324 using a second bond 2326. A rigid, annular inner retainer ring 2328 is affixed perpendicularly across the distal end of a rigid inner tube portion 2330 using a third bond 2332. The inner tube portion 2330 has an outer diameter that is less than an inner diameter of the outer tube portion 2324, allowing the distal end of the inner tube portion to be inserted into the proximal end of the outer tube portion. In some embodiments, the inner diameter of the outer tube portion 2324 is 1.9 mm and the inner diameter of the inner tube portion 2330 is 1.6 mm; however, in other embodiments, different sizes can be used. In the illustrated embodiment, the first, second and third bonds 2322, 2326 and 2332 are made using laser welding; however, in other embodiments these bonds can be made using other forms of welding, adhesives or other known bonding or joining methods.

The tunneling probe 2304 further includes a flexible membrane 2024 that is captured between a proximal face of the outer retaining ring 2318 and a distal face of the inner retaining ring 2338 when the inner tube portion 2330 is inserted into the outer tube portion 2324 and affixed together using a fourth bond 2334. In the illustrated embodiment, the fourth bond 2334 is made using laser welding; however, in other embodiments this bond can be made using other forms of welding, adhesives or other known bonding or joining methods. The fourth bond 2334 must be sufficient to provide a fluid seal and a mechanical coupling between the inner and outer tube portions 2330 and 2324; however, it need not extend along the entire longitudinal interface between the tube portions.

In some embodiments, the inner tube portion 2330 is biased against the outer tube portion 2324 in the longitudinal direction until the flexible membrane 2024 is compressed between the inner retaining ring 2328 and outer retaining ring 2318 when the fourth bond 2334 is formed. This results in a permanent mechanical bias squeezing edges of the membrane 2024 to secure it in place. In some embodiments, the flexible membrane 2024 is bonded to the outer retaining ring 2318 using a fifth bond 2336 and/or bonded to the inner retaining ring 2328 using a sixth bond 2338. In the illustrated embodiment, the fifth and sixth bonds 2336 and 2338 are made using adhesive bonding; however, in other embodiments these bonds can be made using welding, thermal bonding or other known bonding or joining methods. In some embodiments, both mechanical biasing and bonding are used to affix the flexible membrane to the can 2312; however, in other embodiments only one of the aforesaid types of affixation is In some embodiments, the inner tube portion 2330 is biased against the outer tube portion 2324 in the longitudinal direction until the flexible membrane 2024 is compressed between the inner retaining ring 2328 and outer retaining ring 2318 when the fourth bond is formed. This results in a permanent mechanical bias squeezing edges of the membrane 2024 to secure it in place. In some embodiments, the flexible membrane 2024 is bonded to the outer retaining ring 2318 using a fifth bond 2336 and/or bonded to the inner retaining ring 2328 using a sixth bond 2338. In the illustrated embodiment, the fifth and sixth bonds 2336 and 2338 are made using adhesive bonding; however, in other embodiments these bonds can be made using welding, thermal bonding or other known bonding or joining methods. In some embodiments, both mechanical biasing and bonding are used to affix the flexible membrane to the can 2312; however, in other embodiments only one of the aforesaid types of affixation are used. used.

The distal end of the catheter shaft 2002 is disposed in the proximal end of the inner tube portion 2330, and a seventh bond 2340 is formed therebetween to affix the tunneling probe 2304 in position on the catheter shaft. In the illustrated embodiment, the seventh bond 2340 is a thermal or adhesive bond; however, in other embodiments the seventh bond can be formed using welding or other known bonding or joining methods. The seventh bond 2340 must be sufficient to provide a fluid seal and a mechanical coupling between the catheter shaft 2002 and the can 2312; however, it need not extend along the entire longitudinal interface between the shaft and the can.

The various rigid and flexible components described herein in connection with tunneling catheter 2300 can be formed from the same materials, and using the same parameters as described for tunneling catheter 2000. Although the bonds 2322, 2326, 2332, 2334, 2336, 2338 and 2340 have been labeled as “first” through “seventh” bonds for convenience, these labels do not indicate any limitations in the order of assembly of the tunneling catheter 2300, and one of ordinary skill will appreciate that various assembly orders are expected. The method of operation of the catheter 2300 for creating modified plaque that is more susceptible to dislocation than the original calcified tissue is similar to that of the tunneling catheters previously described herein.

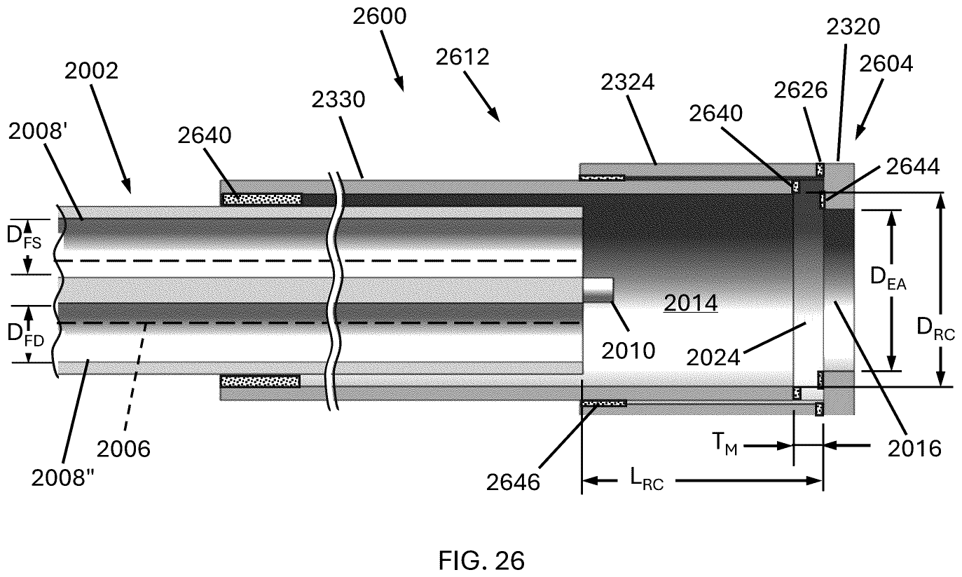

Referring now to FIG. 26, there is illustrated a lithotripsy catheter with a sealed-chamber “drum” type tunneling probe in accordance with yet another embodiment. The embodiment of FIG. 26 is similar to the embodiment shown in FIGS. 23-25, but uses a modified end effector. Unless otherwise described, this embodiment operates in a fashion similar to the embodiment described in connection with FIGS. 23-25, and elements in the description and drawings identified by the same reference numerals are identical or functionally equivalent to those previously described. The tunneling lithotripsy catheter 2600 includes a multi-lumen catheter shaft 2002 and tunneling probe/end effector 2604 affixed on the distal end of the catheter shaft. The end effector 2604 of this embodiment has a different structure for securing the flexible membrane 2024, which includes fewer components and fewer bonds than the previous embodiment.

The multi-lumen catheter shaft 2002 of this tunneling catheter 2600 is substantially similar to those previously described. The tunneling probe 2604 comprises a rigid outer shell or “can” 2612 defining a resonating chamber 2014 therewithin and an emitting aperture 2016 open in the distal direction along the central axis of the catheter shaft. The can 2612 includes a rigid, annular face ring 2320, an outer tube portion 2324 and an inner tube portion 2330 as in the previous embodiment, but it does not require separate inner or outer retaining rings. The face ring 2320 is affixed perpendicularly across the distal end of an outer tube portion 2324 using a first bond 2626. In the illustrated embodiment, the first bond 2626 is made using laser welding; however, in other embodiments this bond can be made using other forms of welding, adhesives or other known bonding or joining methods. As in the previous embodiment, the inner tube portion 2330 has an outer diameter that is less than an inner diameter of the outer tube portion 2324, allowing the distal end of the inner tube portion to be inserted into the proximal end of the outer tube portion.

The tunneling probe 2604 further includes a flexible membrane 2024 that is captured between a proximal face of the face ring 2320 and a distal face of the inner tube portion 2330 when the inner tube portion is inserted into the outer tube portion 2324 and affixed together using a second bond 2640. In the illustrated embodiment, the second bond 2640 is made using laser welding; however, in other embodiments this bond can be made using other forms of welding, adhesives or other known bonding or joining methods. The second bond 2640 must be sufficient to provide a fluid seal and a mechanical coupling between the inner and outer tube portions 2330 and 2324; however, it need not extend along the entire longitudinal interface between the tube portions.

In some embodiments, the inner tube portion 2330 is biased against the outer tube portion 2324 in the longitudinal direction until the flexible membrane 2024 is compressed between the inner tube portion and the face ring 2320 when the second bond 2640 is formed. This results in a permanent mechanical bias squeezing edges of the membrane 2024 to secure it in place. In some embodiments, the flexible membrane 2024 is bonded to the inner tube portion 2330 using a third bond 2640 and/or bonded to the face ring 2320 using a fourth bond 2644. In the illustrated embodiment, the third and fourth bonds 2640 and 2644 are made using laser welding; however, in other embodiments these bonds can be made using other types of welding, thermal bonding, adhesives or other known bonding or joining methods. In some embodiments, both mechanical biasing and bonding are used to affix the flexible membrane to the can 2612; however, in other embodiments only one of the aforesaid types of affixation is used.

The distal end of the catheter shaft 2002 is disposed in the proximal end of the inner tube portion 2330, and a fifth bond 2646 is formed therebetween to affix the tunneling probe 2604 in position on the catheter shaft. In the illustrated embodiment, the fifth bond 2646 is a thermal or adhesive bond; however, in other embodiments the fifth bond can be formed using welding or other known bonding or joining methods. The fifth bond 2646 must be sufficient to provide a fluid seal and a mechanical coupling between the catheter shaft 2002 and the can 2612; however, it need not extend along the entire longitudinal interface between the shaft and the can

The various rigid and flexible components described herein in connection with tunneling catheter 2600 can be formed from the same materials, and using the same parameters as described for tunneling catheter 2000 and 2300. Although the bonds 2626, 2640, 2642 and 2644 have been labeled as “first” through “fourth” bonds for convenience, these labels do not indicate any limitations in the order of assembly of the tunneling catheter 2600, and one of ordinary skill will appreciate that various assembly orders are expected. The method of operation of the catheter 2600 for creating modified plaque that is more susceptible to dislocation than the original calcified tissue is similar to that of the tunneling catheters previously described herein.

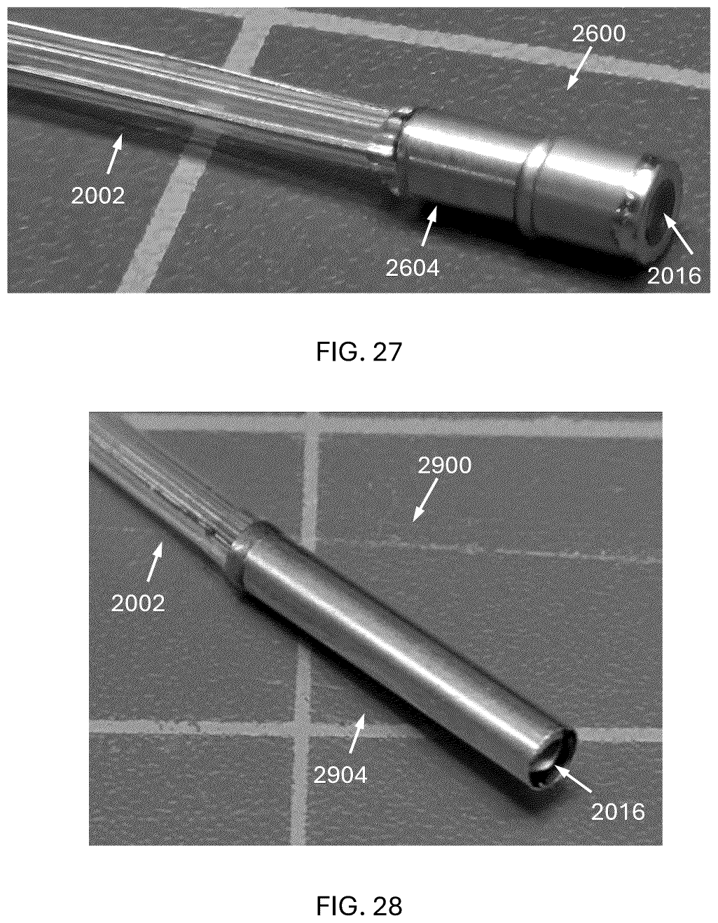

Referring now also to FIG. 27, there is illustrated a prototype tunneling catheter 2600 in accordance with the embodiment of FIG. 26.

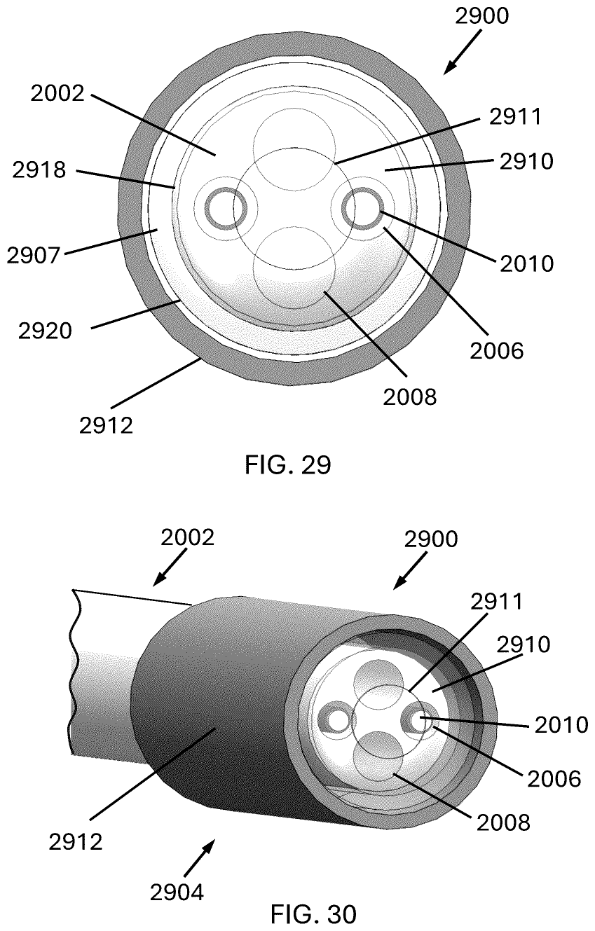

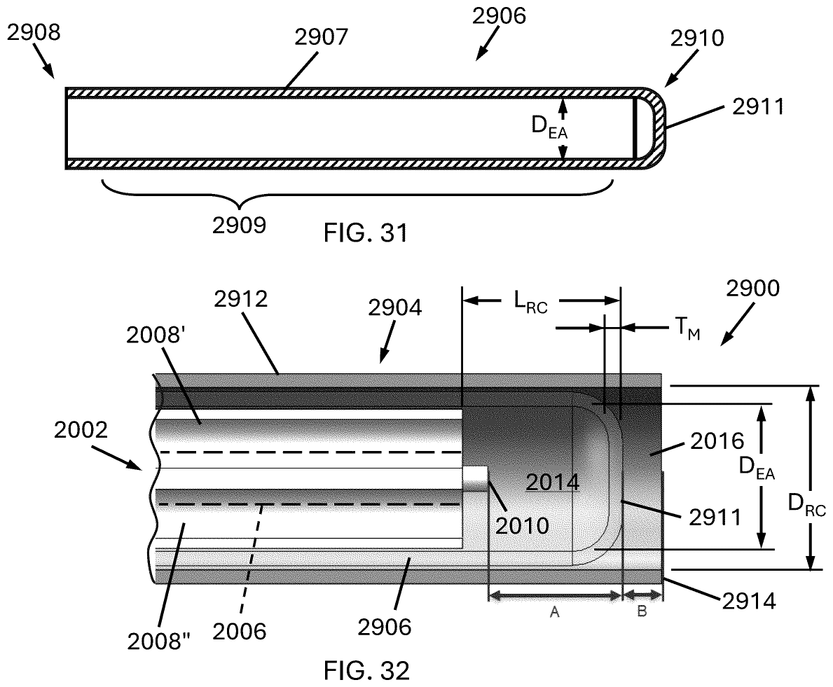

Referring now to FIGS. 28-31; there is illustrated a lithotripsy catheter with a sealed-chamber “sock” type tunneling probe in accordance with yet another embodiment. FIG. 28 illustrates a prototype tunneling catheter 2900 in accordance with this embodiment. As further explained below, instead of using a flat, disk-shaped flexible membrane in the tunneling probe, the tunneling catheter 2900 uses an elongated, tubular, closed-end flexible membrane that resembles a sock (see FIG. 31).

The lithotripsy catheter 2900 includes a multi-lumen catheter shaft 2002 and a tunneling probe/end effector 2904. The multi-lumen catheter shaft 2002 of this embodiment and its constituent elements including electrode lumens 2006, fluid lumens 2008, and electrodes 2010 are substantially similar to those previously described. The function and operation of the catheter shaft 2002 to produce electrically-induced pressure waves is also similar. The tunneling probe 2904 is affixed on the distal end of the catheter shaft 2002 and includes a rigid can 2912 that houses a sock type flexible membrane 2906 (i.e., the “sock”).

Referring now specifically to FIG. 31, a cross-sectional view of the sock membrane 2906 is shown. The sock 2906 includes a tubular sidewall 2907 that extends from an open proximal end portion 2908 (i.e., the “cuff”), along a substantially constant diameter portion 2909 (i.e., the “ankle”) to a closed distal end portion 2910 (i.e., the “toe”). In some embodiments, the toe 2910 includes a flat portion 2011 oriented perpendicular across the longitudinal axis of the sock. In some embodiments, the sidewall 2007 of the sock 2009 has a substantially constant wall thickness (designated T_{M} in FIG. 32). For efficient pressure pulse transmission from the resonating chamber 2014 to the exterior medium having the target calcifications, the sock membrane 2906 should be very soft and/or very thin. In some embodiments, the sock membrane 2906 is formed of an elastomer material having a durometer within the range from 50 Shore A to 35 Shore D. In some preferred embodiments, the sock membrane 2906 is formed of an elastomer material having a durometer within the range from 80 Shore A to 90 Shore A. In some embodiments, the elastomer material of the sock membrane 2906 is a polyurethane material, whereas in other embodiments, other elastomer or polymer materials can be used. In some embodiments, the sock membrane 2906 can be formed by dipping a mandrel in a liquid polymer material, which is then allowed to dry/cure. One dip or multiple dips can be used to achieve the required thickness. In some embodiments, the sock membrane 2906 can be formed by blow-molding a polymer material. Other methods that can be used in other embodiments to make the sock membrane 2906 include dip casting/molding, injection molding, blow molding, extrusion and thermal tip forming.

Referring now specifically to FIG. 32, the sock membrane 2906 is disposed over the distal end of the multi-lumen shaft 2002 to create a first predetermined distance (denoted A in FIG. 32) between the tip of the electrode 2010 and the distal surface of the toe 2911. The rigid can 2912 is disposed over the distal end of the sock membrane 2906 to create a second predetermined distance (denoted B in FIG. 32) between the distal surface of the toe 2911 and the distal end of the can.

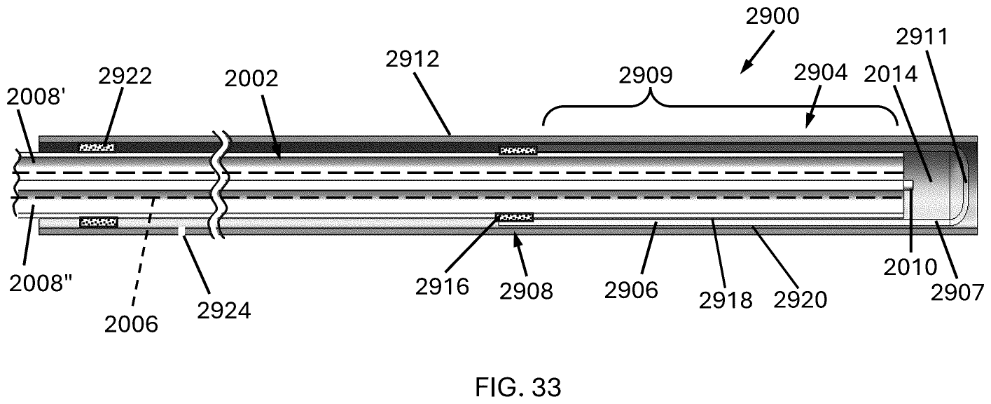

Referring now also to FIG. 33, the sock membrane 2906 can be bonded to the catheter shaft 2002 at the proximal end “cuff” portion 2908 with a first bond 2916, leaving the central “ankle” portion 2909 of the sock membrane unbonded to both the catheter shaft and the rigid can 2912. Thus, extending longitudinally from the distal “toe” end 2910 to the proximal “cuff” end 2908 of the sock membrane 2906 is an annular interior gap 2918 (i.e., between the sock sidewall 2907 and the catheter shaft 2002) and an annular exterior gap 2920 (i.e., between the sock sidewall and the rigid can 2912). The entire unbonded portion of the sock sidewall 2907 along the ankle portion 2909 is thus free to stretch in the longitudinal direction in response to pressure wave induced oscillations in the resonating chamber 2014 while still remaining radially constrained by the rigid can 2912 to prevent sidewall blowouts. Since the unbonded length of the sock membrane 2906 from toe 2010 to first bond 2916 is substantially larger than the length L_{RC} of the resonating chamber 2014, the strain per unit length in the unbonded sidewalls 2907 caused by the oscillations (i.e., elongations) of the distal toe end will be substantially smaller than if the same elongations were experienced only in the sidewalls bordering the resonating chamber. This “damping” of the strains (and associated stresses) in the sock sidewalls 2907 improves the reliability of the tunneling probe 2904.

The distal end of the catheter shaft 2002 is disposed within the can 2912, and optionally, a second bond 2922 can be formed therebetween to affix the can (i.e., outer skin) of the tunneling probe 2904 to the catheter shaft. The second bond 2922 (if present) can be formed anywhere along the shaft 2022, including at the hub (not shown) at the proximal end. In the illustrated embodiment, the second bond 2922 is a thermal or adhesive bond; however, in other embodiments the second bond can be formed using welding or other known bonding or joining methods. When present, the second bond 2922 must be sufficient to provide mechanical coupling between the catheter shaft 2002 and the can 2912; however, a fluid-tight seal is not required because the fluid seal to the catheter shaft 2022 is provided by the first bond 2916. In some embodiments, some or all of the can 2912 disposed proximate from the resonating chamber may be laser cut to provide flexibility for better trackability while still providing hoop strength to radially reinforce the sock membrane 2906 and catheter shaft 2002 against internal pressure.

It is well known that introducing air into the blood stream can be hazardous to patients. It is therefore common practice with catheters and other devices having annular spaces between inner and outer members to flush the annular space with a liquid prior to use to remove any trapped air. In the lithotripsy catheter 2900, the annular exterior gap 2920 between the sock sidewall 2907 and the can 2912 allows flushing the annular spaces between the sock 2906 and outer metal tube 2912 and between the outer metal tube and the shaft 2002 to remove any air prior to use. In embodiments having a second bond 2922 between the outer metal tube 2912 and the shaft 2002, one or more ports 2924 can be provided through the outer metal tube into the annular gap to facilitate air removal during flushing.

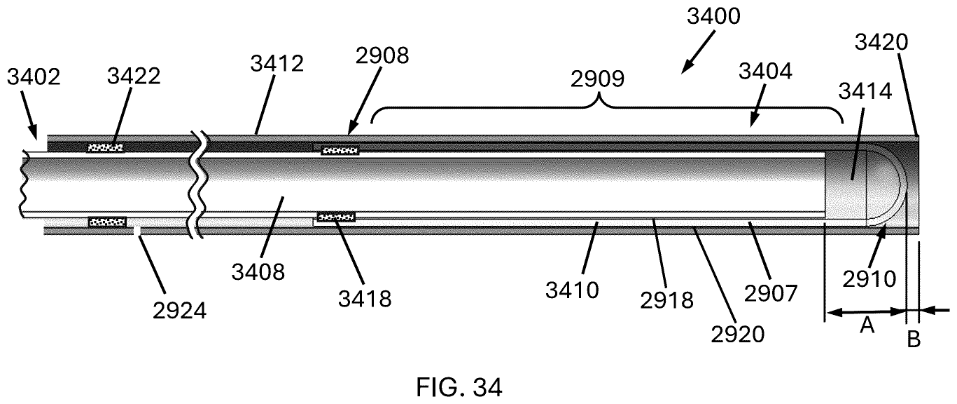

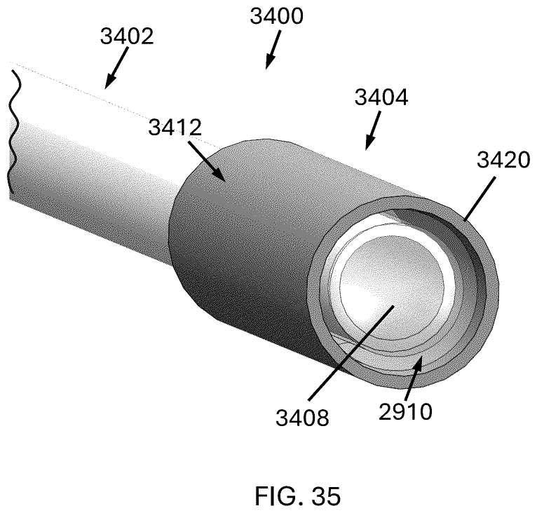

Referring now to FIGS. 34 and 35, there is illustrated a lithotripsy catheter with a sealed-chamber “sock” type tunneling probe in accordance with a further embodiment. In this embodiment, the lithotripsy catheter 3400 has an alternative single-lumen catheter shaft 3402 and a tunneling probe 3404 similar to that described in connection with previous embodiments. This arrangement allows the lithotripsy catheter 3400 to utilize an external high-frequency hydraulic pulsatile pressure wave generator to produce the pressure waves used for creating modified plaques.

The single-lumen catheter shaft 3402 includes a single fluid lumen 3408 and does not require any electrode lumens. The fluid lumen 3408 can be operatively connected to an external high-frequency hydraulic pulsatile pressure wave generator (not shown), for example the Pulse IVI™ system produced by AVS Pulse, Inc., which is powered by carbon dioxide. The output of the pressure wave generator is connected to the proximal end of the catheter shaft 3402 using standard ports or other connection hardware. The pressure waves produced by the pressure wave generator are transmitted by a working fluid, e.g., saline, along the fluid lumen 3408 to the tunneling probe 3404 disposed at the distal end of the catheter shaft.

The tunneling probe 3404 includes a rigid can 3412 (i.e., outer skin) and a sock-type flexible membrane 3410 having a tubular sidewall 2907 that extends from an open proximal end portion 2908 (“cuff”), along a substantially constant diameter portion 2909 (“ankle”) to a closed distal end portion 2910 (“toe”) similar to the sock membrane 2906 previously described. The sock membrane 3410 is disposed over the distal portion of the catheter shaft 3402 to create a first predetermined distance (denoted A in FIG. 34) between the distal end of the catheter shaft and the distal surface of the toe portion 2910, thus defining a pulsate chamber 3414. The sock membrane 3410 is affixed to the catheter shaft 3402 at the proximal end “cuff” portion 2908 with a first bond 3418, leaving the central “ankle” portion 2909 of the sock membrane unbonded to the catheter shaft. The rigid can 3412 is disposed over the distal end of the sock membrane 3410 to create a second predetermined distance (denoted B in FIG. 34) between the distal surface of the toe 2910 and the distal end 3420 of the can. The pulsate chamber 3414 is thereby setback from the distal end 3420 of the can 3412 to protect the flexible membrane 3410 from contact with calcified tissue during operation.

Extending longitudinally from the distal “toe” end 2910 to the proximal “cuff” end 2908 of the sock membrane 3410 is an annular interior gap 2918 (i.e., between the sock sidewall 2907 and the catheter shaft 3402) and an annular exterior gap 2920 (i.e., between the sock sidewall and the rigid can 3412). The entire unbonded portion of the sock sidewall 2907 along the ankle portion 2909 is thus free to stretch in the longitudinal direction in response to pressure wave induced oscillations in the pulsate chamber 3414. Optionally, a second bond 3422 can be provided to affix the can (i.e., outer skin) of the tunneling probe 3404 to the catheter shaft 3402. The second bond 3422 (if present) can be formed anywhere along the shaft 3422, including at the hub (not shown) at the proximal end. In the illustrated embodiment, the second bond 3422 is a thermal or adhesive bond; however, in other embodiments the second bond can be formed using welding or other known bonding or joining methods. When present, the second bond 3422 must be sufficient to provide mechanical coupling between the catheter shaft 3402 and the can 3412; however, a fluid-tight seal is not required because the fluid seal to the catheter shaft is provided by the first bond 3418. In some embodiments, some or all of the can 3412 may be laser cut to provide flexibility for better trackability while still providing hoop strength to radially reinforce the sock membrane 2906 and catheter shaft 2002 against internal pressure. In embodiments having a second bond 3422 between the outer metal tube 3412 and the shaft 3402, one or more ports 2924 can be provided through the outer metal tube into the annular gap to facilitate air removal during flushing.

During operation of the tunneling catheter 3400, hydraulic shock waves from the external pulsatile pressure wave generator travel up the catheter shaft 3402 to the pulsate chamber 3414 and are focused axially forward through the flexible membrane 3410 of the tunneling probe and subsequently propagate into calcified tissue. Repeated exposure of the calcified tissue to hydraulic shock waves creates modified plaque that is more susceptible to dislocation than the original calcified tissue. Additional embodiments are contemplated within the scope of the current invention, e.g., the tunneling catheters 900, 2000, 2300, 2600 and 2900 can be adapted to use an external pulsatile pressure wave generator by substituting a single lumen catheter shaft for the respective multi-lumen catheter shaft in each embodiment described.

Although the preferred embodiment has been described in detail, it should be understood that various changes, substitutions and alterations can be made therein without departing from the spirit and scope of the invention as defined by the appended claims.

What is claimed is:

1. A lithotripsy device for tunneling through vascular occlusions of calcified tissue in a liquid environment of a blood vessel, the device comprising:
a tunneling catheter including
a tunneling catheter shaft having a distal end and a proximal end;
a tunneling probe disposed on the distal end of the tunneling catheter shaft, the tunneling probe having a pair of electrodes;
each electrode being operatively connected to an electrical lead within the tunneling catheter shaft;
wherein the leads are configured for operative connection to an external pulse generator;
wherein activation of an external pulse generator operatively connected to the leads causes repeated electrical discharges between the electrodes;
wherein, when the electrodes are an in a fluid, the repeated electrical discharges produce repeated hydraulic shock waves in the fluid directed away from the electrical discharges;
wherein, when the repeated hydraulic shock waves strike calcified tissue, the calcified tissue is broken into a modified plaque; and
wherein the modified plaque has a lower resistance to mechanical dislocation that the original calcified tissue.

2. A lithotripsy device in accordance with claim 1, wherein the tunneling probe is configured with a raised rim on the distal end; and
wherein the electrodes are inset within the raised rim.

3. A lithotripsy device in accordance with claim 2, wherein the electrodes are configured in a concentric arrangement within the raised rim.

4. A lithotripsy device in accordance with claim 2, wherein the electrodes are configured in an opposing arcuate arrangement within the raised rim.

5. A lithotripsy device in accordance with claim 1, wherein the tunneling probe is configured with a tapering conical face on the distal end; and
wherein the electrodes are configured in a spaced-apart arrangement on the tapering conical face.

6. A lithotripsy device in accordance with claim 5, wherein the tapering conical face of the tunneling probe terminates in a flat face; and
wherein the electrodes are exposed on the flat face.

7. A lithotripsy device in accordance with claim 5, wherein the tapering conical face of the tunneling probe terminates in a rounded end; and
wherein the electrodes are not exposed on the rounded end.

8. A lithotripsy device in accordance with claim 1, further comprising a guidewire lumen formed through the catheter shaft and having an exit on the distal end; and
wherein the tunneling probe is configured with a flat face on the distal end; and
wherein the electrodes protrude above the flat face.

9. A lithotripsy device in accordance with claim 8, wherein the exit for the guidewire lumen is disposed in the center of the flat face of the distal end and the electrodes are configured in an opposing arcuate arrangement on opposite sides of the exit.

10. A lithotripsy device in accordance with claim 8, wherein the exit for the guidewire lumen is disposed offset from the center of the flat face of the distal end and the electrodes are configured as studs adjacent to one another on one side of the exit.

11. A lithotripsy device in accordance with claim 8, wherein the exit for the guidewire lumen is disposed in the center of the flat face of the distal end and the electrodes are configured as studs on opposite sides of the exit.

12. A lithotripsy device in accordance with claim 1, further comprising a positioning balloon operatively attached to the tunneling catheter shaft near the distal end; and
wherein the positioning balloon can be selectively inflated to position the catheter tunneling probe at a desired location within a cross section of the vessel.

13. A lithotripsy device in accordance with claim 1, further comprising:
a support catheter comprising a support catheter shaft having a distal end and a proximal end and defining an interior lumen configured to accommodate the tunneling catheter passing therethrough;
wherein, when the support catheter is positioned with the distal end near the occlusion of calcified tissue, the proximal end can be affixed at an introduction site; and
wherein, when the support catheter is affixed at the introduction site, the tunneling catheter can be selectively inserted and withdrawn through the interior lumen to reach the occlusion of calcified tissue with the distal end having the tunneling probe.

14. A lithotripsy device in accordance with claim 13, further comprising a positioning balloon operatively attached to the support catheter shaft near the distal end; and
wherein the positioning balloon can be selectively inflated to position the distal end of the support catheter at a desired location within a cross section of the vessel; and
wherein, when the tunneling catheter is introduced through the support catheter, the tunneling probe of the tunneling catheter will emerge from the support catheter at the desired location withing the cross section of the vessel.

15. A lithotripsy device in accordance with claim 1, further comprising a pulse generator operatively connected to the electrical leads.

16. A lithotripsy device for tunneling through vascular occlusions of calcified tissue in a liquid environment of a blood vessel, the device comprising:
a tunneling catheter including
a tunneling catheter shaft having a distal end and a proximal end;
a tunneling probe disposed on the distal end of the tunneling catheter shaft, the tunneling probe including
a pair of electrodes, each electrode being operatively connected to an electrical lead within the tunneling catheter shaft;
a raised rim on the distal end, wherein the electrodes are inset within the raised rim; and
a flexible diaphragm affixed across the raised rim;
wherein the raised rim and the flexible diaphragm define a probe cavity containing the electrodes, the probe cavity being fluidly isolated from a fluid environment external to the probe cavity;
wherein the leads are configured for operative connection to an external pulse generator;
wherein activation of an external pulse generator operatively connected to the leads causes repeated electrical discharges between the electrodes;
wherein, when the probe cavity contains a working fluid, the repeated electrical discharges produce repeated primary shock waves in the working fluid that repeatedly vibrate the flexible diaphragm;
wherein the repeated vibration of the flexible diaphragm produces repeated secondary shock waves propagated into the fluid environment external to the probe cavity; and
wherein, when the repeated secondary shock waves strike calcified tissue in the fluid environment, the calcified tissue breaks into a modified plaque; and
wherein the modified plaque has a lower resistance to mechanical dislocation that the original calcified tissue.

17. A lithotripsy device in accordance with claim 16, wherein the flexible diaphragm is formed of a metal or metal alloy.

18. A lithotripsy device in accordance with claim 16, wherein the flexible diaphragm is formed of a plastic, polymer, elastomer or other non-metallic material.

19. A lithotripsy device in accordance with claim 16, further comprising a fluid lumen formed within the tunneling catheter shaft and fluidly connecting the probe cavity to a source of fresh working fluid for circulating the fresh working fluid through the probe cavity during the repeated electrical discharges.

20. A method for using a lithotripsy device for tunneling through vascular occlusions of calcified tissue in a liquid environment of a blood vessel, the method comprising the following steps:
providing a lithotripsy tunneling catheter and a support catheter;
introducing a guidewire into a fluid-filled vascular system and guiding the guidewire into a blood vessel having a calcified tissue occlusion;
introducing the support catheter into the vascular system over the guidewire to the location of the calcified tissue occlusion;
inflating a positioning balloon on the support catheter in the blood vessel;
affixing the support catheter at introduction site to minimize axial movement of support catheter in the vessel;
withdrawing the guidewire from the lumen of the support catheter;
introducing the tunneling catheter through support catheter to location of calcified tissue occlusion and positioning the tunneling probe in the blood vessel adjacent the calcified tissue;
producing electrical discharges between electrodes on the tunneling probe to create hydraulic shock waves in the fluid of the vessel;
directing the shock waves produced by the electrical discharges through the fluid in the vessel into the calcified tissue of occlusion to break up the calcified tissue into a modified plaque; and
channeling through the modified plaque with reduced mechanical force to create a channel through the calcified tissue of the occlusion along a desired line.
